(19) United States
(12) Patent Application Publication
(10) Pub. No.: US 2025/0376520 A1
(43) Pub. Date: Dec. 11, 2025

(54) Anti-TRPM4 Antibody For Treating Stroke

(71) Applicant: Singapore Health Services Pte Ltd

(72) Inventor: Ping Liao, Singapore (SG)

(21) Appl. No.: 18/909,785

(22) Filed: Oct. 8, 2024

(30) Foreign Application Priority Data
Jun. 10, 2024 (SG) 10202401671W

(51) Int. Cl. C07K 16/28; A61K 39/00; A61K 45/06; A61P 9/10

(52) U.S. Cl. CPC C07K 16/28; A61K 45/06; A61P 9/10; A61K 2039/505; C07K 2317/24; C07K 2317/565; C07K 2317/76

(57) ABSTRACT

Disclosed are anti-transient receptor potential melastatin 4 (TRPM4) antibodies and their use for treating stroke. In particular, disclosed are humanized monoclonal antibodies specific to TRPM4 and their use for treating stroke.

### CROSS-REFERENCE TO RELATED APPLICATIONS

This application claims the benefit of priority of Singapore Provisional application Ser. No. 10202401671W filed on Jun. 10, 2024, which is incorporated by reference herein in its entirety for any purpose.

### SEQUENCE LISTING

This application contains a Sequence Listing that has been submitted electronically as an XML file named 88771US_Sequence Listing_ST26.xml. The XML file, created on Oct. 7, 2024, is 76,007 bytes in size. The material in the XML file is hereby incorporated by reference in its entirety.

### FIELD OF INVENTION

The present disclosure relates to anti-transient receptor potential melastatin 4 (TRPM4) antibodies and their use for treating stroke. In particular, the present disclosure relates to humanized monoclonal antibodies specific to TRPM4 and their use for treating stroke.

### BACKGROUND

The transient receptor potential melastatin-like subfamily member 4 (TRPM4) is a nonselective cation channel, which conducts or is permeable to monovalent ions such as sodium. TRPM4 channel has been identified as the major pathway for sodium entry in neurons and vascular endothelial cells under hypoxia. Importantly, TRPM4 is activated by ATP depletion and an increase of intracellular Ca²⁺, which are important pathological features associated with hypoxia. TRPM4 activity is greatly enhanced in stroke, and TRPM4 expression is upregulated in surviving neurons and vascular endothelial cells close to the infarct core. As a result, sodium influx via TRPM4 induces cell swelling in neurons and vascular endothelial cells. Therefore, blocking TRPM4 could reduce oncotic cell death in stroke.

Stroke is a leading cause of death worldwide and always results in serious long-term disability. There are two types of stroke: ischemic and hemorrhagic. As the major type of stroke, ischemic stroke occurs when a cerebral artery is blocked by a clot. In the US, 87% stroke cases are ischemic. Currently, the only potent treatment for acute ischemic stroke is reperfusion therapy by restoring blood flow to the affected brain tissue that is still viable.

In stroke, apoptosis and oncosis are common types of cell death. Compared to programmed cell death (apoptosis), cell swelling or oncotic cell death (oncosis) takes place much earlier after stroke onset. Cell swelling (increase in cell volume) is a result of water accumulation inside the cell, which is a passive process that follows the osmotic gradient established by the influx of ions. In healthy cells, active transportation of ions establishes an ionic gradient across cytoplasmic membrane. During stroke, ATP-dependant pumps fail to function due to oxygen and glucose depletion. Meanwhile, ions continue to flux into cells down their respective ionic gradient, which increases intracellular osmotic pressure accordingly. Extracellular sodium (140-145 mM) and chloride (110 mM) have much higher concentrations than calcium (1.8 mM), thus playing a major role in elevating intracellular osmotic pressure. Inside the cell, there are abundant negatively-charged proteins which are unable to cross cell membrane. Therefore, intracellular positively-charged potassium ions, albeit being present at a high concentration, are largely retained inside the cell to balance the negatively-charged proteins. Overall, a net influx of sodium and chloride drives the cell to swell. Therefore, if the pathways for ionic influx are blocked, oncotic cell death can be mitigated and the vascular cell death can be postponed.

The only FDA-approved reperfusion drug for acute ischemic stroke is tissue plasminogen activator (tPA), which has a thrombolytic effect to break up blood clots. Some stroke patients are also eligible for thrombectomy to remove blood clots inside large arteries via endovascular surgery. The major challenge for reperfusion therapy is the very narrow time window. For tPA, the patients can only receive the drug within 4.5 hours after the first symptoms appear. By taking into consideration the time needed for traveling to hospital and diagnosis through brain imaging, majority of stroke patients could not receive reperfusion therapy, and are left without effective treatment. Reperfusion after the time window can still recanalize the blocked vessel. However, as the blood vessels are weakened after sustained hypoxia, they are not able to resist the impact from reperfusion. Therefore, delayed reperfusion often results in severe hemorrhage and edema due to vascular injury. This type of damage is known as reperfusion injury.

In view of the above-mentioned challenges in stroke, there is a need for a novel way to manage vascular and neural protection for stroke. In particular, there is a need to develop new molecules (such as antibodies) and methods to manage and treat stroke.

### SUMMARY

In one aspect, the present disclosure refers to a method of treating stroke, comprising administering to a subject an effective amount of a humanized monoclonal antibody or antigen-binding fragment thereof specific to a transient receptor potential melastatin 4 (TRPM4) protein, wherein the antibody or antigen-binding fragment thereof comprises a heavy chain variable region and a light chain variable region, wherein the heavy chain variable region comprises a CDR1-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 1, a CDR2-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 2 and a CDR3-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 3, and wherein the light chain variable region comprises a CDR1-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 4, a CDR2-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 5 and a CDR3-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 6.

Disclosed is use of an effective amount of a humanized monoclonal antibody or antigen-binding fragment thereof specific to a transient receptor potential melastatin 4 (TRPM4) protein in the manufacture of a medicament for treating stroke, wherein the antibody or antigen-binding fragment thereof comprises a heavy chain variable region and a light chain variable region, wherein the heavy chain variable region comprises a CDR1-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 1, a CDR2-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 2 and a CDR3-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 3, and wherein the light chain variable region comprises a CDR1-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 4, a CDR2-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 5 and a CDR3-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 6.

### BRIEF DESCRIPTION OF DRAWINGS

The present disclosure will be better understood with reference to the detailed description when considered in conjunction with the non-limiting examples and the accompanying drawings, in which:

FIGS. 1A, 1B, 1C, 1D, 1E, and 1F show the evaluation of the in vivo role of M4M in a stroke reperfusion animal model. FIG. 1A shows the experimental protocol for 3-hr transient middle cerebral artery occlusion (MCAO). M4M (100 μg) or control mouse IgG (mIgG, 100 μg) was injected intravenously 1 hr before recanalization. FIG. 1B shows representative images of 2,3,4-triphenyltetrazolium chloride (TTC)-stained rat brains at 1 day after transient MCAO induction. FIG. 1C shows the summary of infarct volume formation. n=6 rats except for M4M and mIgG treatments in heterozygous rats (n=7). FIG. 1D shows the comparison of infarct volume formation after M4M treatment among homozygous (−/−), heterozygous (+/−), and wild-type (+/+) rats. FIG. 1E shows the assessment of motor functions by Rotarod test in homozygous rats after the treatment of M4M (n=7), mIgG (n=6), or vehicle (n=6). FIG. 1F shows the comparison of vehicle (n=6), mIgG (n=6), and M4M (n=7) treatments on the neurological severity scores in homozygous rats. In FIGS. 1C, 1E, and 1F, statistical analysis was performed by two-way ANOVA with Bonferroni post hoc test, and in FIG. 1D by one-way two-way ANOVA with Bonferroni post hoc test. * P<0.05, ** P<0.01. In FIGS. 1E and 1F, significance was observed in M4M vs vehicle or mIgG at day 1. After day 1, significance was found only in M4M vs mIgG.

FIGS. 2A, 2B and 2C illustrate the characterization of humanized anti-TRPM4 antibodies. FIG. 2A shows the immunofluorescent staining of humanized antibodies in HEK 293 cells transfected with human TRPM4. Human IgG was transfected in HEK 293 as control. Antibody clone A1 is an anti-TRPM4 chimeric antibody. Antibody clones A2-A7 are humanized anti-TRPM4 antibodies. Myc staining showed successful transfection of human TRPM4 and Wheat germ agglutinin (WGA) was used as surface glycoproteins staining. FIG. 2B is a bar graph showing the electrophysiological characterization of the chimeric antibody clone (A1) and the humanized antibodies (A2-A7). TRPM4 currents at 0 min and 7 min hypoxia were recorded. Currents at +80 mV and −80 mV were summarized. FIG. 2C is a dose-response curve showing dose-dependent inhibition of humanized antibody clone A6 on hypoxia-induced current increase. IC₅₀: 1.04 μg/ml.

FIGS. 3A, 3B and 3C illustrate the characterization of humanized antibody clone A6 (M4H) in an animal model of stroke reperfusion. MCAO was induced in rats for 3 hours followed by reperfusion. Antibody was delivered 1 hr before reperfusion. FIG. 3A shows representative images of TTC-stained brains from stroke rats receiving 1 mg/kg human IgG or M4H. FIG. 3B shows the summary of infarct volume in rats receiving human IgG or M4H. FIG. 3C shows the assessment of motor functions by Rotarod test in stroke rats receiving human IgG or M4H. * p<0.05, ** p<0.01, *** p<0.001.

FIG. 4 shows sensor-gram plots showing the binding affinity and kinetics of different humanized antibodies to Peptide-biotin. The overlaid smooth black lines represent the kinetic fit of the binding response signals at different antibody concentrations to a 1:1 interaction model.

FIG. 5 shows the cerebral blood flow monitored across different time points in a permanent stroke animal model treated with 1 mg/kg human IgG or M4H. * p<0.05.

FIGS. 6A and 6B show the in vitro ADCC activity of humanized antibody clones A6 (M4H), A6-1, A6-2 and A6-3 across different cell lines. FIG. 6A shows the in vitro ADCC activity of these clones against Human Lung Microvascular Endothelial cells (HULEC-5a). FIG. 6B shows the in vitro ADCC activity of these clones against Human Umbilical Vein Endothelial cells (HUVEC).

### DETAILED DESCRIPTION

The present disclosure describes humanized monoclonal antibodies specific to TRPM4 and their use for treating stroke. The use of humanized antibodies specific to human TRPM4 is expected to demonstrate enhanced therapeutic effects especially in human patients due to lower risk of immune rejection compared to using rabbit or mouse antibodies known in the art.

In one example, the present disclosure refers to a humanized monoclonal antibody or antigen-binding fragment thereof specific to a transient receptor potential melastatin 4 (TRPM4) protein, wherein the antibody or antigen-binding fragment thereof comprises a heavy chain variable region and a light chain variable region, wherein the heavy chain variable region comprises a CDR1-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 1, a CDR2-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 2 and a CDR3-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 3, and wherein the light chain variable region comprises a CDR1-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 4, a CDR2-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 5 and a CDR3-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 6.

In one example, the present disclosure refers to a humanized monoclonal antibody or antigen-binding fragment thereof specific to a transient receptor potential melastatin 4 (TRPM4) protein, wherein the antibody or antigen-binding fragment thereof comprises a heavy chain variable region and a light chain variable region, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 7, SEQ ID NO: 8, SEQ ID NO: 9, or SEQ ID NO:10, and wherein the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 11, SEQ ID NO: 12, SEQ ID NO: 13, or SEQ ID NO: 14. In one example, each heavy and light chain comprises one variable region and one constant region. It is known in the art that the variable region of the heavy and light chain region of an antibody is for antigen binding. In one example, the variable region comprises three complementarity-determining regions (i.e., CDR1, CDR2 and CDR3) and four framework regions (i.e., FR1, FR2, FR3 and FR4). In one example, the complementarity-determining regions and framework regions are in the order of FR1-CDR1-FR2-CDR2-FR3-CDR3-FR4.

In one example, the present disclosure refers to an antibody or antigen-binding fragment thereof disclosed herein, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 11. In one example, the present disclosure refers to an antibody or antigen-binding fragment thereof disclosed herein, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 12. In one example, the present disclosure refers to an antibody or antigen-binding fragment thereof disclosed herein, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 13. In one example, the present disclosure refers to an antibody or antigen-binding fragment thereof disclosed herein, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 14. In one example, the present disclosure refers to an antibody or antigen-binding fragment thereof disclosed herein, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 11. In one example, the present disclosure refers to an antibody or antigen-binding fragment thereof disclosed herein, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 12.

In one example, an antibody or antigen-binding fragment thereof having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11 corresponds to humanized antibody clone A2. In one example, an antibody or antigen-binding fragment thereof having a heavy chain variable region comprising an amino acid sequence of SEQ ID NO: 7, and a light chain variable region comprising an amino acid sequence of SEQ ID NO: 11 corresponds to humanized antibody clone A2. In one example, an antibody or antigen-binding fragment thereof having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12 corresponds to humanized antibody clone A3. In one example, an antibody or antigen-binding fragment thereof having a heavy chain variable region comprising an amino acid sequence of SEQ ID NO: 7, and a light chain variable region comprising an amino acid sequence of SEQ ID NO: 12 corresponds to humanized antibody clone A3. In one example, an antibody or antigen-binding fragment thereof having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 13 corresponds to humanized antibody clone A4. In one example, an antibody or antigen-binding fragment thereof having a heavy chain variable region comprising an amino acid sequence of SEQ ID NO: 7, and a light chain variable region comprising an amino acid sequence of SEQ ID NO: 13 corresponds to humanized antibody clone A4. In one example, an antibody or antigen-binding fragment thereof having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 14 corresponds to humanized antibody clone A5. In one example, an antibody or antigen-binding fragment thereof having a heavy chain variable region comprising an amino acid sequence of SEQ ID NO: 7, and a light chain variable region comprising an amino acid sequence of SEQ ID NO: 14 corresponds to humanized antibody clone A5. In one example, an antibody or antigen-binding fragment thereof having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11 corresponds to humanized antibody clone A6. In one example, an antibody or antigen-binding fragment thereof having a heavy chain variable region comprising an amino acid sequence of SEQ ID NO: 8, and a light chain variable region comprising an amino acid sequence of SEQ ID NO: 11 corresponds to humanized antibody clone A6. In one example, an antibody or antigen-binding fragment thereof having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11 corresponds to humanized antibody clone A6-1. In one example, an antibody or antigen-binding fragment thereof having a heavy chain variable region comprising an amino acid sequence of SEQ ID NO: 8, and a light chain variable region comprising an amino acid sequence of SEQ ID NO: 11 corresponds to humanized antibody clone A6-1. In one example, an antibody or antigen-binding fragment thereof having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11 corresponds to humanized antibody clone A6-2. In one example, an antibody or antigen-binding fragment thereof having a heavy chain variable region comprising an amino acid sequence of SEQ ID NO: 8, and a light chain variable region comprising an amino acid sequence of SEQ ID NO: 11 corresponds to humanized antibody clone A6-2. In one example, an antibody or antigen-binding fragment thereof having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11 corresponds to humanized antibody clone A6-3. In one example, an antibody or antigen-binding fragment thereof having a heavy chain variable region comprising an amino acid sequence of SEQ ID NO: 8, and a light chain variable region comprising an amino acid sequence of SEQ ID NO: 11 corresponds to humanized antibody clone A6-3. In one example, an antibody or antigen-binding fragment thereof having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12 corresponds to humanized antibody clone A7. In one example, an antibody or antigen-binding fragment thereof having a heavy chain variable region comprising an amino acid sequence of SEQ ID NO: 8, and a light chain variable region comprising an amino acid sequence of SEQ ID NO: 12 corresponds to humanized antibody clone A7.

In one example, the present disclosure refers to an antibody or antigen-binding fragment thereof disclosed herein, wherein the antibody or antigen-binding fragment thereof comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, SEQ ID NO: 51, SEQ ID NO: 52, SEQ ID NO: 53, SEQ ID NO: 54, SEQ ID NO. 55, or SEQ ID NO: 56.

In one example, the present disclosure refers to an antibody or antigen-binding fragment thereof disclosed herein, wherein the antibody or antigen-binding fragment thereof comprises a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57, SEQ ID NO: 58, SEQ ID NO: 59, or SEQ ID NO: 60.

In one example, the present disclosure refers to an antibody or antigen-binding fragment thereof disclosed herein, wherein the antibody or antigen-binding fragment thereof comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, SEQ ID NO: 51, SEQ ID NO: 52, SEQ ID NO: 53, SEQ ID NO: 54, SEQ ID NO: 55, or SEQ ID NO: 56, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57, SEQ ID NO: 58, SEQ ID NO: 59, or SEQ ID NO: 60.

In one example, the present disclosure refers to an antibody or antigen-binding fragment thereof disclosed herein, wherein the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 57. In one example, the present disclosure refers to an antibody or antigen-binding fragment thereof disclosed herein, wherein the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 58. In one example, the present disclosure refers to an antibody or antigen-binding fragment thereof disclosed herein, wherein the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 59. In one example, the present disclosure refers to an antibody or antigen-binding fragment thereof disclosed herein, wherein the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 60. In one example, the present disclosure refers to an antibody or antigen-binding fragment thereof disclosed herein, wherein the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 57. In one example, the present disclosure refers to an antibody or antigen-binding fragment thereof disclosed herein, wherein the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 58. In one example, the present disclosure refers to an antibody or antigen-binding fragment thereof disclosed herein, wherein the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 54, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 57. In one example, the present disclosure refers to an antibody or antigen-binding fragment thereof disclosed herein, wherein the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 55, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 57. In one example, the present disclosure refers to an antibody or antigen-binding fragment thereof disclosed herein, wherein the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 56, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 57.

In one example, an antibody or antigen-binding fragment thereof having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57 corresponds to humanized antibody clone A2. In one example, an antibody or antigen-binding fragment thereof having a heavy chain comprising an amino acid sequence of SEQ ID NO: 50, and a light chain comprising an amino acid sequence of SEQ ID NO: 57 corresponds to humanized antibody clone A2. In one example, an antibody or antigen-binding fragment thereof having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 58 corresponds to humanized antibody clone A3. In one example, an antibody or antigen-binding fragment thereof having a heavy chain comprising an amino acid sequence of SEQ ID NO: 50, and a light chain comprising an amino acid sequence of SEQ ID NO: 58 corresponds to humanized antibody clone A3. In one example, an antibody or antigen-binding fragment thereof having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 59 corresponds to humanized antibody clone A4. In one example, an antibody or antigen-binding fragment thereof having a heavy chain comprising an amino acid sequence of SEQ ID NO: 50, and a light chain comprising an amino acid sequence of SEQ ID NO: 59 corresponds to humanized antibody clone A4. In one example, an antibody or antigen-binding fragment thereof having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 60 corresponds to humanized antibody clone A5. In one example, an antibody or antigen-binding fragment thereof having a heavy chain comprising an amino acid sequence of SEQ ID NO: 50, and a light chain comprising an amino acid sequence of SEQ ID NO: 60 corresponds to humanized antibody clone A5. In one example, an antibody or antigen-binding fragment thereof having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57 corresponds to humanized antibody clone A6. In one example, an antibody or antigen-binding fragment thereof having a heavy chain comprising an amino acid sequence of SEQ ID NO: 51, and a light chain comprising an amino acid sequence of SEQ ID NO: 57 corresponds to humanized antibody clone A6. In one example, an antibody or antigen-binding fragment thereof having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 58 corresponds to humanized antibody clone A7. In one example, an antibody or antigen-binding fragment thereof having a heavy chain comprising an amino acid sequence of SEQ ID NO: 51, and a light chain comprising an amino acid sequence of SEQ ID NO: 58 corresponds to humanized antibody clone A7. In one example, an antibody or antigen-binding fragment thereof having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 54, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57 corresponds to humanized antibody clone A6-1. In one example, an antibody or antigen-binding fragment thereof having a heavy chain comprising an amino acid sequence of SEQ ID NO: 54, and a light chain comprising an amino acid sequence of SEQ ID NO: 57 corresponds to humanized antibody clone A6-1. In one example, an antibody or antigen-binding fragment thereof having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 55, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57 corresponds to humanized antibody clone A6-2. In one example, an antibody or antigen-binding fragment thereof having a heavy chain comprising an amino acid sequence of SEQ ID NO: 55, and a light chain comprising an amino acid sequence of SEQ ID NO: 57 corresponds to humanized antibody clone A6-2. In one example, an antibody or antigen-binding fragment thereof having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 56, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57 corresponds to humanized antibody clone A6-3. In one example, an antibody or antigen-binding fragment thereof having a heavy chain comprising an amino acid sequence of SEQ ID NO: 56, and a light chain comprising an amino acid sequence of SEQ ID NO: 57 corresponds to humanized antibody clone A6-3.

It is known in the art that the constant region of the heavy and light chain region of an antibody comprises a more conserved amino acid sequence compared to the variable region. In one example, the antibody or antigen-binding fragment disclosed herein comprises a constant region selected from the major classes of immunoglobulins, such as IgG, IgD, IgE, IgA and IgM. In one example, the constant region is selected from the group consisting of IgG1, IgG2, IgG3, and IgG4. In one example, the constant region is IgG1. In another example, the constant region is IgG4. In one example, the antibody or antigen-binding fragment thereof disclosed herein comprises a heavy chain comprising an IgG1 constant region, where said heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO; 50, SEQ ID NO: 51, SEQ ID NO: 52, SEQ ID NO: 53, SEQ ID NO: 54 or SEQ ID NO: 55. In one example, the antibody or antigen-binding fragment thereof disclosed herein comprises a heavy chain comprising an IgG4 constant region, where said heavy chain comprises an amino acid sequence having at least 85%, 86%, 87%, 88%, 89%, 90%, 91%, 92%, 93%, 94%, 95%, 96%, 97%, 98%, or 99% sequence identity SEQ ID NO: 56.

In one example, the present disclosure refers to an antibody or antigen-binding fragment thereof disclosed herein, wherein the antibody specifically binds to a peptide comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 15; or a peptide comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 16.

In one example, the antibody or antigen-binding fragment thereof disclosed herein inhibits TRPM4 activity. In one example, the antibody or antigen-binding fragment thereof disclosed herein inhibits TRPM4 activity by inhibiting TRPM4 current. In one example, the antibody or antigen-binding fragment thereof disclosed herein inhibits TRPM4 activity by internalizing membrane TRPM4 protein. In one example, the antibody or antigen-binding fragment thereof disclosed herein inhibits TRPM4 activity by inhibiting TRPM4 current and internalizing membrane TRPM4 protein. In one example, under diseased conditions, inhibiting TRPM4 activity refers to blocking, preventing or disrupting the upregulated or activated protein function of TRPM4.

In one example, the concentration of the antibody or antigen-binding fragment thereof required to inhibit TRPM4 activity in vitro is from 1 μg/ml to 5 μg/ml, or from 2 μg/ml to 4 μg/ml, or about 1 μg/ml, or about 2 μg/ml, or about 3 μg/ml, or about 4 μg/ml, or about 5 μg/ml. In one example, the concentration of the antibody or antigen-binding fragment thereof required to inhibit TRPM4 activity in vitro is 1.23 μg/ml. In one example, the concentration of the antibody or antigen-binding fragment thereof required to inhibit TRPM4 activity in vitro is 1.04 μg/ml. In one example, the concentration of the antibody or antigen-binding fragment thereof required to inhibit TRPM4 activity in vitro may be determined by a dose-dependent assay detecting for the dose-dependent effect of the antibody or antigen-binding fragment thereof on hypoxia-induced TRPM4 current increase.

In one example, the present disclosure refers to a nucleic acid encoding the antibody or antigen-binding fragment thereof disclosed herein. In one example, the nucleic acid comprises a polynucleotide sequence having at least 85%, 86%, 87%, 88%, 89%, 90%, 91%, 92%, 93%, 94%, 95%, 96%, 97%, 98%, or 99% sequence identity to any one of SEQ ID NOs: 17-24 and SEQ ID NOs: 61-63. In one example, the heavy chain and the light chain of the antibody or antigen-binding fragment thereof disclosed herein are encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 21, respectively. In one example, the heavy chain and the light chain of the antibody or antigen-binding fragment thereof disclosed herein are encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 22, respectively. In one example, the heavy chain and the light chain of the antibody or antigen-binding fragment thereof disclosed herein are encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 23, respectively. In one example, the heavy chain and the light chain of the antibody or antigen-binding fragment thereof disclosed herein are encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 24, respectively. In one example, the heavy chain and the light chain of the antibody or antigen-binding fragment thereof disclosed herein are encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 18 and 21, respectively. In one example, the heavy chain and the light chain of the antibody or antigen-binding fragment thereof disclosed herein are encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 18 and 22, respectively. In one example, the heavy chain of the antibody or antigen-binding fragment thereof disclosed herein is encoded by a polynucleotide sequence having at least 85%, 86%, 87%, 88%, 89%, 90%, 91%, 92%, 93%, 94%, 95%, 96%, 97%, 98%, or 99% sequence identity to SEQ ID NO: 61. In one example, the heavy chain of the antibody or antigen-binding fragment thereof disclosed herein is encoded by a polynucleotide sequence having at least 85%, 86%, 87%, 88%, 89%, 90%, 91%, 92%, 93%, 94%, 95%, 96%, 97%, 98%, or 99% sequence identity to SEQ ID NO: 62. In one example, the heavy chain of the antibody or antigen-binding fragment thereof disclosed herein is encoded by a polynucleotide sequence having at least 85%, 86%, 87%, 88%, 89%, 90%, 91%, 92%, 93%, 94%, 95%, 96%, 97%, 98%, or 99% sequence identity to SEQ ID NO: 63. In one example, the heavy chain and the light chain of the antibody or antigen-binding fragment thereof disclosed herein are encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 61 and 21, respectively. In one example, the heavy chain and the light chain of the antibody or antigen-binding fragment thereof disclosed herein are encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 62 and 21, respectively. In one example, the heavy chain and the light chain of the antibody or antigen-binding fragment thereof disclosed herein are encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 63 and 21, respectively.

In one example, an antibody or antigen-binding fragment thereof having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 21, respectively corresponds to humanized antibody clone A2. In one example, an antibody or antigen-binding fragment thereof having its heavy chain and its light chain encoded by polynucleotide sequences of SEQ ID NOs: 17 and 21, respectively, corresponds to humanized antibody clone A2. In one example, an antibody or antigen-binding fragment thereof having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 22, respectively corresponds to humanized antibody clone A3. In one example, an antibody or antigen-binding fragment thereof having its heavy chain and its light chain encoded by polynucleotide sequences of SEQ ID NOs: 17 and 22, respectively, corresponds to humanized antibody clone A3. In one example, an antibody or antigen-binding fragment thereof having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 23, respectively corresponds to humanized antibody clone A4. In one example, an antibody or antigen-binding fragment thereof having its heavy chain and its light chain encoded by polynucleotide sequences of SEQ ID NOs: 17 and 23, respectively, corresponds to humanized antibody clone A4. In one example, an antibody or antigen-binding fragment thereof having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 24, respectively corresponds to humanized antibody clone A5. In one example, an antibody or antigen-binding fragment thereof having its heavy chain and its light chain encoded by polynucleotide sequences of SEQ ID NOs: 17 and 24, respectively, corresponds to humanized antibody clone A5. In one example, an antibody or antigen-binding fragment thereof having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 18 and 21, respectively corresponds to humanized antibody clone A6. In one example, an antibody or antigen-binding fragment thereof having its heavy chain and its light chain encoded by polynucleotide sequences of SEQ ID NOs: 18 and 21, respectively, corresponds to humanized antibody clone A6. In one example, an antibody or antigen-binding fragment thereof having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 18 and 22, respectively corresponds to humanized antibody clone A7. In one example, an antibody or antigen-binding fragment thereof having its heavy chain and its light chain encoded by polynucleotide sequences of SEQ ID NOs: 18 and 22, respectively, corresponds to humanized antibody clone A7. In one example, an antibody or antigen-binding fragment thereof having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 61 and 21, respectively corresponds to humanized antibody clone A6-1. In one example, an antibody or antigen-binding fragment thereof having its heavy chain and its light chain encoded by polynucleotide sequences of SEQ ID NOs: 61 and 21, respectively, corresponds to humanized antibody clone A6-1. In one example, an antibody or antigen-binding fragment thereof having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 62 and 21, respectively corresponds to humanized antibody clone A6-2. In one example, an antibody or antigen-binding fragment thereof having its heavy chain and its light chain encoded by polynucleotide sequences of SEQ ID NOs: 62 and 21, respectively, corresponds to humanized antibody clone A6-2. In one example, an antibody or antigen-binding fragment thereof having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 63 and 21, respectively corresponds to humanized antibody clone A6-3. In one example, an antibody or antigen-binding fragment thereof having its heavy chain and its light chain encoded by polynucleotide sequences of SEQ ID NOs: 63 and 21, respectively, corresponds to humanized antibody clone A6-3.

In one example, the present disclosure refers to an expression vector comprising the nucleic acid disclosed herein. Suitable expression vectors for production of recombinant proteins (such as antibodies, such as the humanized antibodies of the present disclosure) are well known to those skilled in the art and examples of expression vectors comprising the nucleic acid disclosed herein include pcDNA3.1, pGEX and pCMV.

In one example, the present disclosure refers to a host cell comprising the nucleic acid disclosed herein. In one example, the present disclosure refers to a host cell comprising the expression vector disclosed herein. In one example, the present disclosure refers to a host cell comprising the nucleic acid disclosed herein and the expression vector disclosed herein. In one example, the host cell comprising the nucleic acid disclosed herein and/or the expression vector disclosed herein is human embryonic kidney 293 cells (HEK 293 cells) or Chinese hamster ovary cells (CHO cells). In one example, the host cell comprising the nucleic acid disclosed herein and/or the expression vector disclosed herein may be cells generally used for the production of humanized antibodies which are known to those skilled in the art.

In one example, the present disclosure refers to a method of producing the antibody or antigen-binding fragment thereof disclosed herein. In one example, the method comprises culturing the host cell disclosed herein in a culture medium. In one example, the culture medium is Dulbecco's Modified Eagle's Medium (DMEM) (12800017; Thermo Fisher Scientific, USA) supplemented with 10% fetal bovine serum (10500064, Thermo Fisher Scientific, USA); 1.74 g/L sodium bicarbonate; 1.2 g/L HEPES; and 100 U/mL Penicillin-Streptomycin (15140122; Thermo Fisher Scientific, USA), or any other suitable cell culture medium known in the art. In one example, the method further comprises isolating the antibody or antigen-binding fragment thereof from the culture medium. In one example, the method of producing and isolating the antibody or antigen-binding fragment thereof disclosed herein may be methods generally used for the production of recombinant proteins, such as humanized antibodies, which are known to those skilled in the art.

In one example, point mutation(s) has been made to the antibody or antigen-binding fragment thereof disclosed herein to stabilize the resulting antibody clone. In one example, at least one point mutation(s) has been made in a framework region(s) of the antibody to stabilize the resulting antibody clone. In one example, one point mutation has been made in a framework region(s) of the antibody to stabilize the resulting antibody clone. In one example, two point mutations have been made in a framework region(s) of the antibody to stabilize the resulting antibody clone. In one example, three point mutations have been made in a framework region(s) of the antibody to stabilize the resulting antibody clone. In one example, the at least one point mutation(s) can be made in FR1, FR2, FR3 or FR4, or combinations thereof, of the framework region of the antibody. In one example, one point mutation has been made in FR2 of the framework region of the antibody. In one example, two point mutations have been made in FR2 of the framework region of the antibody. In one example, one point mutation has been made in FR3 of the framework region of the antibody. In one example, two point mutations have been made in FR3 of the framework region of the antibody. In one example, three point mutations have been made in FR3 of the framework region of the antibody. In one example, there is no upper limit in the number of mutations made to a framework region of the antibody. In one example, one or more point mutations can be made in any framework region of any heavy chain variable region (such as the heavy chain variable region comprising an amino acid having at least 85%, 86%, 87%, 88%, 89%, 90%, 91%, 92%, 93%, 94%, 95%, 96%, 97%, 98%, or 99% sequence identity to SEQ ID NO: 7, SEQ ID NO: 8, SEQ ID NO: 9, or SEQ ID NO: 10) or any light chain variable region (such as the light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11, SEQ ID NO: 12, SEQ ID NO: 13, or SEQ ID NO: 14). In one example, the term “point mutation” refers to a mutation where a single amino acid is substituted, inserted or deleted from an amino acid sequence.

In another example, modification(s) can be made to a constant region of the heavy chain and/or light chain to reduce toxicity. In one example, the modification(s) is point mutation(s). In one example, point mutation(s) has been made to the constant region of the heavy chain of the antibody or antigen-binding fragment thereof disclosed herein to reduce toxicity. In one example, at least one point mutation(s) has been made in the constant region of the heavy chain (or heavy chain constant region) of the antibody to reduce toxicity. In one example, one point mutation has been made in the heavy chain constant region of the antibody to reduce toxicity. In one example, two point mutations have been made in the heavy chain constant region of the antibody to reduce toxicity. In one example, the at least one point mutation(s) can be made in the IgG1, IgG2, IgG3 or IgG4 heavy chain constant region of the antibody or antigen-binding fragment thereof disclosed herein to reduce toxicity. In one example, the at least one point mutation(s) can be made in the IgG1, IgG2, IgG3 or IgG4 heavy chain constant region of the antibody or antigen-binding fragment thereof disclosed herein to reduce cytotoxicity such as antibody-dependent cellular cytotoxicity (ADCC), antibody-dependent cellular phagocytosis (ADCP), and/or complement-dependent cytotoxicity (CDC). In one example, the at least one point mutation(s) can be made in the IgG1 heavy chain constant region of the antibody or antigen-binding fragment thereof disclosed herein to reduce toxicity. In one example, the at least one point mutation(s) can be made in the IgG4 heavy chain constant region of the antibody or antigen-binding fragment thereof disclosed herein to reduce toxicity. In one example, one point mutation has been made in the IgG1 heavy chain constant region of the antibody to reduce toxicity. In one example, two point mutations have been made in the IgG1 heavy chain constant region of the antibody to reduce toxicity. In one example, one or two point mutations have been made in the IgG1 heavy chain constant region of the antibody to reduce cytotoxicity such as ADCC, ADCP, and/or CDC. In one example, one point mutation has been made in the IgG4 heavy chain constant region of the antibody to reduce toxicity. In one example, one point mutation has been made in the IgG4 heavy chain constant region of the antibody to reduce cytotoxicity such as ADCC, ADCP, and/or CDC. In one example, there is no upper limit in the number of point mutations made to a heavy chain constant region of the antibody. In one example, the LL residues at positions 261 and 262 of IgG1 heavy chain constant region (e.g. positions 261 and 262 of IgG1 heavy chain constant region of VH5) are substituted with AA residues. In one example, substitution of LL residues at positions 261 and 262 of IgG1 heavy chain constant region (e.g. positions 261 and 262 of IgG1 heavy chain constant region of VH5) with AA residues results in a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 54. In one example, the N residue at position 324 of IgG1 heavy chain constant region (e.g. position 324 of IgG1 heavy chain constant region of VH6) is substituted with an A residue. In one example, substitution of N residue at position 324 of IgG1 heavy chain constant region (e.g. position 324 of IgG1 heavy chain constant region of VH6) with an A residue results in a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 55. In one example, the S residue at position 254 of IgG4 heavy chain constant region (e.g. position 254 of IgG4 heavy chain constant region of VH7) is substituted with a P residue. In one example, substitution of S residue at position 254 of IgG4 heavy chain constant region (e.g. position 254 of IgG4 heavy chain constant region of VH7) with a P residue results in a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 56. The modifications disclosed herein can reduce toxicity while maintaining the antigen-binding activity of the variable region of the antibody (or fragment thereof) such that it retains similar and/or identical binding affinity.

In one example, the present disclosure refers to a pharmaceutical composition comprising the antibody or antigen-binding fragment thereof disclosed herein, and a pharmaceutically acceptable carrier, excipient, or diluent. In one example, the present disclosure refers to the antibody or antigen-binding fragment thereof disclosed herein for use as a medicament.

In one example, the present disclosure refers to a method of treating stroke, comprising administering to a subject an effective amount of the antibody or antigen-binding fragment thereof disclosed herein, or the pharmaceutical composition disclosed herein. Therefore, in one aspect, the present disclosure refers to a method of treating stroke, comprising administering to a subject an effective amount of a humanized monoclonal antibody or antigen-binding fragment thereof specific to a transient receptor potential melastatin 4 (TRPM4) protein, wherein the antibody or antigen-binding fragment thereof comprises a heavy chain variable region and a light chain variable region, wherein the heavy chain variable region comprises a CDR1-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 1, a CDR2-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 2 and a CDR3-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 3, and wherein the light chain variable region comprises a CDR1-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 4, a CDR2-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 5 and a CDR3-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 6. In one example, the antibody or antigen-binding fragment thereof for treating stroke comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, SEQ ID NO: 8, SEQ ID NO: 9, or SEQ ID NO: 10, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11, SEQ ID NO: 12, SEQ ID NO: 13, or SEQ ID NO: 14. In one example of the method disclosed herein, each heavy and light chain of the antibody or antigen-binding fragment thereof comprises one variable region and one constant region. It is known in the art that the variable region of the heavy and light chain region of an antibody is for antigen binding. In one example of the method disclosed herein, the variable region comprises three complementarity-determining regions (i.e., CDR1, CDR2 and CDR3) and four framework regions (i.e., FR1, FR2, FR3 and FR4). In one example of the method disclosed herein, the complementarity-determining regions and framework regions are in the order of FR1-CDR1-FR2-CDR2-FR3-CDR3-FR4.

In one example, the present disclosure refers to use of an effective amount of the antibody or antigen-binding fragment thereof disclosed herein, or the pharmaceutical composition disclosed herein, in the manufacture of a medicament for treating stroke. Therefore, in one aspect, the present disclosure refers to use of an effective amount of a humanized monoclonal antibody or antigen-binding fragment thereof specific to a transient receptor potential melastatin 4 (TRPM4) protein in the manufacture of a medicament for treating stroke, wherein the antibody or antigen-binding fragment thereof comprises a heavy chain variable region and a light chain variable region, wherein the heavy chain variable region comprises a CDR1-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 1, a CDR2-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 2 and a CDR3-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 3, and wherein the light chain variable region comprises a CDR1-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 4, a CDR2-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 5 and a CDR3-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 6. In one example, the antibody or antigen-binding fragment thereof for use in the manufacture of a medicament for treating stroke comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, SEQ ID NO: 8, SEQ ID NO: 9, or SEQ ID NO: 10, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11, SEQ ID NO: 12, SEQ ID NO: 13, or SEQ ID NO: 14. In one example of the use disclosed herein, each heavy and light chain of the antibody or antigen-binding fragment thereof comprises one variable region and one constant region. It is known in the art that the variable region of the heavy and light chain region of an antibody is for antigen binding. In one example of the use disclosed herein, the variable region comprises three complementarity-determining regions (i.e., CDR1, CDR2 and CDR3) and four framework regions (i.e., FR1, FR2, FR3 and FR4). In one example of the use disclosed herein, the complementarity-determining regions and framework regions are in the order of FR1-CDR1-FR2-CDR2-FR3-CDR3-FR4.

In one example, the antibody or antigen-binding fragment thereof for treating stroke or for use in the manufacture of a medicament for treating stroke comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11. In one example, the antibody or antigen-binding fragment thereof for treating stroke or for use in the manufacture of a medicament for treating stroke comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12. In one example, the antibody or antigen-binding fragment thereof for treating stroke or for use in the manufacture of a medicament for treating stroke comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 13. In one example, the antibody or antigen-binding fragment thereof for treating stroke or for use in the manufacture of a medicament for treating stroke comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 14. In one example, the antibody or antigen-binding fragment thereof for treating stroke or for use in the manufacture of a medicament for treating stroke comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11. In one example, the antibody or antigen-binding fragment thereof for treating stroke or for use in the manufacture of a medicament for treating stroke comprises a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12.

In one example, the antibody or antigen-binding fragment thereof for treating stroke or for use in the manufacture of a medicament for treating stroke, having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11 corresponds to humanized antibody clone A2. In one example, the antibody or antigen-binding fragment thereof for treating stroke or for use in the manufacture of a medicament for treating stroke, having a heavy chain variable region comprising an amino acid sequence of SEQ ID NO: 7, and a light chain variable region comprising an amino acid sequence of SEQ ID NO: 11 corresponds to humanized antibody clone A2. In one example, the antibody or antigen-binding fragment thereof for treating stroke or for use in the manufacture of a medicament for treating stroke, having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12 corresponds to humanized antibody clone A3. In one example, the antibody or antigen-binding fragment thereof for treating stroke or for use in the manufacture of a medicament for treating stroke, having a heavy chain variable region comprising an amino acid sequence of SEQ ID NO: 7, and a light chain variable region comprising an amino acid sequence of SEQ ID NO: 12 corresponds to humanized antibody clone A3. In one example, the antibody or antigen-binding fragment thereof for treating stroke or for use in the manufacture of a medicament for treating stroke, having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 13 corresponds to humanized antibody clone A4. In one example, the antibody or antigen-binding fragment thereof for treating stroke or for use in the manufacture of a medicament for treating stroke, having a heavy chain variable region comprising an amino acid sequence of SEQ ID NO: 7, and a light chain variable region comprising an amino acid sequence of SEQ ID NO: 13 corresponds to humanized antibody clone A4. In one example, the antibody or antigen-binding fragment thereof for treating stroke or for use in the manufacture of a medicament for treating stroke, having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 14 corresponds to humanized antibody clone A5. In one example, the antibody or antigen-binding fragment thereof for treating stroke or for use in the manufacture of a medicament for treating stroke, having a heavy chain variable region comprising an amino acid sequence of SEQ ID NO: 7, and a light chain variable region comprising an amino acid sequence of SEQ ID NO: 14 corresponds to humanized antibody clone A5. In one example, the antibody or antigen-binding fragment thereof for treating stroke or for use in the manufacture of a medicament for treating stroke, having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11 corresponds to humanized antibody clone A6. In one example, the antibody or antigen-binding fragment thereof for treating stroke or for use in the manufacture of a medicament for treating stroke, having a heavy chain variable region comprising an amino acid sequence of SEQ ID NO: 8, and a light chain variable region comprising an amino acid sequence of SEQ ID NO: 11 corresponds to humanized antibody clone A6. In one example, the antibody or antigen-binding fragment thereof for treating stroke or for use in the manufacture of a medicament for treating stroke, having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11 corresponds to humanized antibody clone A6-1. In one example, the antibody or antigen-binding fragment thereof for treating stroke or for use in the manufacture of a medicament for treating stroke, having a heavy chain variable region comprising an amino acid sequence of SEQ ID NO: 8, and a light chain variable region comprising an amino acid sequence of SEQ ID NO: 11 corresponds to humanized antibody clone A6-1. In one example, the antibody or antigen-binding fragment thereof for treating stroke or for use in the manufacture of a medicament for treating stroke, having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11 corresponds to humanized antibody clone A6-2. In one example, the antibody or antigen-binding fragment thereof for treating stroke or for use in the manufacture of a medicament for treating stroke, having a heavy chain variable region comprising an amino acid sequence of SEQ ID NO: 8, and a light chain variable region comprising an amino acid sequence of SEQ ID NO: 11 corresponds to humanized antibody clone A6-2. In one example, the antibody or antigen-binding fragment thereof for treating stroke or for use in the manufacture of a medicament for treating stroke, having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11 corresponds to humanized antibody clone A6-3. In one example, the antibody or antigen-binding fragment thereof for treating stroke or for use in the manufacture of a medicament for treating stroke, having a heavy chain variable region comprising an amino acid sequence of SEQ ID NO: 8, and a light chain variable region comprising an amino acid sequence of SEQ ID NO: 11 corresponds to humanized antibody clone A6-3. In one example, the antibody or antigen-binding fragment thereof for treating stroke or for use in the manufacture of a medicament for treating stroke, having a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12 corresponds to humanized antibody clone A7. In one example, the antibody or antigen-binding fragment thereof for treating stroke or for use in the manufacture of a medicament for treating stroke, having a heavy chain variable region comprising an amino acid sequence of SEQ ID NO: 8, and a light chain variable region comprising an amino acid sequence of SEQ ID NO: 12 corresponds to humanized antibody clone A7.

In one example, the antibody or antigen-binding fragment thereof for treating stroke or for use in the manufacture of a medicament for treating stroke comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, SEQ ID NO: 51, SEQ ID NO: 52, SEQ ID NO: 53, SEQ ID NO: 54, SEQ ID NO. 55, or SEQ ID NO: 56.

In one example, the antibody or antigen-binding fragment thereof for treating stroke or for use in the manufacture of a medicament for treating stroke comprises a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57, SEQ ID NO: 58, SEQ ID NO: 59, or SEQ ID NO: 60.

In one example, the antibody or antigen-binding fragment thereof for treating stroke or for use in the manufacture of a medicament for treating stroke comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, SEQ ID NO: 51, SEQ ID NO: 52, SEQ ID NO: 53, SEQ ID NO: 54, SEQ ID NO: 55, or SEQ ID NO: 56, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57, SEQ ID NO: 58, SEQ ID NO: 59, or SEQ ID NO: 60.

In one example, the antibody or antigen-binding fragment thereof for treating stroke or for use in the manufacture of a medicament for treating stroke comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57. In one example, the antibody or antigen-binding fragment thereof for treating stroke or for use in the manufacture of a medicament for treating stroke comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 58. In one example, the antibody or antigen-binding fragment thereof for treating stroke or for use in the manufacture of a medicament for treating stroke comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 59. In one example, the antibody or antigen-binding fragment thereof for treating stroke or for use in the manufacture of a medicament for treating stroke comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 60. In one example, the antibody or antigen-binding fragment thereof for treating stroke or for use in the manufacture of a medicament for treating stroke comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57. In one example, the antibody or antigen-binding fragment thereof for treating stroke or for use in the manufacture of a medicament for treating stroke comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 58. In one example, the antibody or antigen-binding fragment thereof for treating stroke or for use in the manufacture of a medicament for treating stroke comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 54, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57. In one example, the antibody or antigen-binding fragment thereof for treating stroke or for use in the manufacture of a medicament for treating stroke comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 55, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57. In one example, the antibody or antigen-binding fragment thereof for treating stroke or for use in the manufacture of a medicament for treating stroke comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 56, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57.

In one example, the antibody or antigen-binding fragment thereof for treating stroke or for use in the manufacture of a medicament for treating stroke, having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57 corresponds to humanized antibody clone A2. In one example, the antibody or antigen-binding fragment thereof for treating stroke or for use in the manufacture of a medicament for treating stroke, having a heavy chain comprising an amino acid sequence of SEQ ID NO: 50, and a light chain comprising an amino acid sequence of SEQ ID NO: 57 corresponds to humanized antibody clone A2. In one example, the antibody or antigen-binding fragment thereof for treating stroke or for use in the manufacture of a medicament for treating stroke, having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 58 corresponds to humanized antibody clone A3. In one example, the antibody or antigen-binding fragment thereof for treating stroke or for use in the manufacture of a medicament for treating stroke, having a heavy chain comprising an amino acid sequence of SEQ ID NO: 50, and a light chain comprising an amino acid sequence of SEQ ID NO: 58 corresponds to humanized antibody clone A3. In one example, the antibody or antigen-binding fragment thereof for treating stroke or for use in the manufacture of a medicament for treating stroke, having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 59 corresponds to humanized antibody clone A4. In one example, the antibody or antigen-binding fragment thereof for treating stroke or for use in the manufacture of a medicament for treating stroke, having a heavy chain comprising an amino acid sequence of SEQ ID NO: 50, and a light chain comprising an amino acid sequence of SEQ ID NO: 59 corresponds to humanized antibody clone A4. In one example, the antibody or antigen-binding fragment thereof for treating stroke or for use in the manufacture of a medicament for treating stroke, having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 60 corresponds to humanized antibody clone A5. In one example, the antibody or antigen-binding fragment thereof for treating stroke or for use in the manufacture of a medicament for treating stroke, having a heavy chain comprising an amino acid sequence of SEQ ID NO: 50, and a light chain comprising an amino acid sequence of SEQ ID NO: 60 corresponds to humanized antibody clone A5. In one example, the antibody or antigen-binding fragment thereof for treating stroke or for use in the manufacture of a medicament for treating stroke, having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57 corresponds to humanized antibody clone A6. In one example, the antibody or antigen-binding fragment thereof for treating stroke or for use in the manufacture of a medicament for treating stroke, having a heavy chain comprising an amino acid sequence of SEQ ID NO: 51, and a light chain comprising an amino acid sequence of SEQ ID NO: 57 corresponds to humanized antibody clone A6. In one example, the antibody or antigen-binding fragment thereof for treating stroke or for use in the manufacture of a medicament for treating stroke, having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 58 corresponds to humanized antibody clone A7. In one example, the antibody or antigen-binding fragment thereof for treating stroke or for use in the manufacture of a medicament for treating stroke, having a heavy chain comprising an amino acid sequence of SEQ ID NO: 51, and a light chain comprising an amino acid sequence of SEQ ID NO: 58 corresponds to humanized antibody clone A7. In one example, the antibody or antigen-binding fragment thereof for treating stroke or for use in the manufacture of a medicament for treating stroke, having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 54, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57 corresponds to humanized antibody clone A6-1. In one example, the antibody or antigen-binding fragment thereof for treating stroke or for use in the manufacture of a medicament for treating stroke, having a heavy chain comprising an amino acid sequence of SEQ ID NO: 54, and a light chain comprising an amino acid sequence of SEQ ID NO: 57 corresponds to humanized antibody clone A6-1. In one example, the antibody or antigen-binding fragment thereof for treating stroke or for use in the manufacture of a medicament for treating stroke, having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 55, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57 corresponds to humanized antibody clone A6-2. In one example, the antibody or antigen-binding fragment thereof for treating stroke or for use in the manufacture of a medicament for treating stroke, having a heavy chain comprising an amino acid sequence of SEQ ID NO: 55, and a light chain comprising an amino acid sequence of SEQ ID NO: 57 corresponds to humanized antibody clone A6-2. In one example, the antibody or antigen-binding fragment thereof for treating stroke or for use in the manufacture of a medicament for treating stroke, having a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 56, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57 corresponds to humanized antibody clone A6-3. In one example, the antibody or antigen-binding fragment thereof for treating stroke or for use in the manufacture of a medicament for treating stroke, having a heavy chain comprising an amino acid sequence of SEQ ID NO: 56, and a light chain comprising an amino acid sequence of SEQ ID NO: 57 corresponds to humanized antibody clone A6-3.

In one example, the antibody or antigen-binding fragment thereof for treating stroke or for use in the manufacture of a medicament for treating stroke comprises a constant region selected from the major classes of immunoglobulins, such as IgG, IgD, IgE, IgA and IgM. In one example, the constant region is selected from the group consisting of IgG1, IgG2, IgG3, and IgG4. In one example, the constant region is IgG1. In another example, the constant region is IgG4. In one example, the antibody or antigen-binding fragment thereof for treating stroke or for use in the manufacture of a medicament for treating stroke comprises a heavy chain comprising an IgG1 constant region, wherein said heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO; 50, SEQ ID NO: 51, SEQ ID NO: 52, SEQ ID NO: 53, SEQ ID NO: 54 or SEQ ID NO: 55. In one example, the antibody or antigen-binding fragment thereof for treating stroke or for use in the manufacture of a medicament for treating stroke comprises a heavy chain comprising an IgG4 constant region, wherein said heavy chain comprises an amino acid sequence having at least 85%, 86%, 87%, 88%, 89%, 90%, 91%, 92%, 93%, 94%, 95%, 96%, 97%, 98%, or 99% sequence identity SEQ ID NO: 56.

In one example, the antibody or antigen-binding fragment thereof for treating stroke or for use in the manufacture of a medicament for treating stroke specifically binds to a peptide comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 15; or a peptide comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 16.

In one example, the antibody or antigen-binding fragment thereof for treating stroke or for use in the manufacture of a medicament for treating stroke is encoded by a nucleic acid comprising a polynucleotide sequence having at least 85%, 86%, 87%, 88%, 89%, 90%, 91%, 92%, 93%, 94%, 95%, 96%, 97%, 98%, or 99% sequence identity to any one of SEQ ID NOs: 17-24 and SEQ ID NOs: 61-63. In one example, the antibody or antigen-binding fragment thereof for treating stroke or for use in the manufacture of a medicament for treating stroke, comprises a heavy chain and a light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 21, respectively. In one example, the antibody or antigen-binding fragment thereof for treating stroke or for use in the manufacture of a medicament for treating stroke, comprises a heavy chain and a light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 22, respectively. In one example, the antibody or antigen-binding fragment thereof for treating stroke or for use in the manufacture of a medicament for treating stroke, comprises a heavy chain and a light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 23, respectively. In one example, the antibody or antigen-binding fragment thereof for treating stroke or for use in the manufacture of a medicament for treating stroke, comprises a heavy chain and a light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 24, respectively. In one example, the antibody or antigen-binding fragment thereof for treating stroke or for use in the manufacture of a medicament for treating stroke, comprises a heavy chain and a light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 18 and 21, respectively. In one example, the antibody or antigen-binding fragment thereof for treating stroke or for use in the manufacture of a medicament for treating stroke, comprises a heavy chain and a light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 18 and 22, respectively. In one example, the antibody or antigen-binding fragment thereof for treating stroke or for use in the manufacture of a medicament for treating stroke, comprises a heavy chain encoded by a polynucleotide sequence having at least 85%, 86%, 87%, 88%, 89%, 90%, 91%, 92%, 93%, 94%, 95%, 96%, 97%, 98%, or 99% sequence identity to SEQ ID NO: 61. In one example, the antibody or antigen-binding fragment thereof for treating stroke or for use in the manufacture of a medicament for treating stroke, comprises a heavy chain encoded by a polynucleotide sequence having at least 85%, 86%, 87%, 88%, 89%, 90%, 91%, 92%, 93%, 94%, 95%, 96%, 97%, 98%, or 99% sequence identity to SEQ ID NO: 62. In one example, the antibody or antigen-binding fragment thereof for treating stroke or for use in the manufacture of a medicament for treating stroke, comprises a heavy chain encoded by a polynucleotide sequence having at least 85%, 86%, 87%, 88%, 89%, 90%, 91%, 92%, 93%, 94%, 95%, 96%, 97%, 98%, or 99% sequence identity to SEQ ID NO: 63. In one example, the antibody or antigen-binding fragment thereof for treating stroke or for use in the manufacture of a medicament for treating stroke, comprises a heavy chain and a light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 61 and 21, respectively. In one example, the antibody or antigen-binding fragment thereof for treating stroke or for use in the manufacture of a medicament for treating stroke, comprises a heavy chain and a light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 62 and 21, respectively. In one example, the antibody or antigen-binding fragment thereof for treating stroke or for use in the manufacture of a medicament for treating stroke, comprises a heavy chain and a light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 63 and 21, respectively.

In one example, the antibody or antigen-binding fragment thereof for treating stroke or for use in the manufacture of a medicament for treating stroke, having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 21, respectively corresponds to humanized antibody clone A2. In one example, the antibody or antigen-binding fragment thereof for treating stroke or for use in the manufacture of a medicament for treating stroke, having its heavy chain and its light chain encoded by polynucleotide sequences of SEQ ID NOs: 17 and 21, respectively, corresponds to humanized antibody clone A2. In one example, the antibody or antigen-binding fragment thereof for treating stroke or for use in the manufacture of a medicament for treating stroke, having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 22, respectively corresponds to humanized antibody clone A3. In one example, the antibody or antigen-binding fragment thereof for treating stroke or for use in the manufacture of a medicament for treating stroke, having its heavy chain and its light chain encoded by polynucleotide sequences of SEQ ID NOs: 17 and 22, respectively, corresponds to humanized antibody clone A3. In one example, the antibody or antigen-binding fragment thereof for treating stroke or for use in the manufacture of a medicament for treating stroke, having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 23, respectively corresponds to humanized antibody clone A4. In one example, the antibody or antigen-binding fragment thereof for treating stroke or for use in the manufacture of a medicament for treating stroke, having its heavy chain and its light chain encoded by polynucleotide sequences of SEQ ID NOs: 17 and 23, respectively, corresponds to humanized antibody clone A4. In one example, the antibody or antigen-binding fragment thereof for treating stroke or for use in the manufacture of a medicament for treating stroke, having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 17 and 24, respectively corresponds to humanized antibody clone A5. In one example, the antibody or antigen-binding fragment thereof for treating stroke or for use in the manufacture of a medicament for treating stroke, having its heavy chain and its light chain encoded by polynucleotide sequences of SEQ ID NOs: 17 and 24, respectively, corresponds to humanized antibody clone A5. In one example, the antibody or antigen-binding fragment thereof for treating stroke or for use in the manufacture of a medicament for treating stroke, having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 18 and 21, respectively corresponds to humanized antibody clone A6. In one example, the antibody or antigen-binding fragment thereof for treating stroke or for use in the manufacture of a medicament for treating stroke, having its heavy chain and its light chain encoded by polynucleotide sequences of SEQ ID NOs: 18 and 21, respectively, corresponds to humanized antibody clone A6. In one example, the antibody or antigen-binding fragment thereof for treating stroke or for use in the manufacture of a medicament for treating stroke, having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 18 and 22, respectively corresponds to humanized antibody clone A7. In one example, the antibody or antigen-binding fragment thereof for treating stroke or for use in the manufacture of a medicament for treating stroke, having its heavy chain and its light chain encoded by polynucleotide sequences of SEQ ID NOs: 18 and 22, respectively, corresponds to humanized antibody clone A7. In one example, the antibody or antigen-binding fragment thereof for treating stroke or for use in the manufacture of a medicament for treating stroke, having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 61 and 21, respectively corresponds to humanized antibody clone A6-1. In one example, the antibody or antigen-binding fragment thereof for treating stroke or for use in the manufacture of a medicament for treating stroke, having its heavy chain and its light chain encoded by polynucleotide sequences of SEQ ID NOs: 61 and 21, respectively, corresponds to humanized antibody clone A6-1. In one example, the antibody or antigen-binding fragment thereof for treating stroke or for use in the manufacture of a medicament for treating stroke, having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 62 and 21, respectively corresponds to humanized antibody clone A6-2. In one example, the antibody or antigen-binding fragment thereof for treating stroke or for use in the manufacture of a medicament for treating stroke, having its heavy chain and its light chain encoded by polynucleotide sequences of SEQ ID NOs: 62 and 21, respectively, corresponds to humanized antibody clone A6-2. In one example, the antibody or antigen-binding fragment thereof for treating stroke or for use in the manufacture of a medicament for treating stroke, having its heavy chain and its light chain encoded by polynucleotide sequences having at least 85%, 86%, 87%, 88%, 89%, 90%, 91%, 92%, 93%, 94%, 95%, 96%, 97%, 98%, or 99% sequence identity to SEQ ID NOs: 63 and 21, respectively corresponds to humanized antibody clone A6-3. In one example, the antibody or antigen-binding fragment thereof for treating stroke or for use in the manufacture of a medicament for treating stroke, having its heavy chain and its light chain encoded by polynucleotide sequences of SEQ ID NOs: 63 and 21, respectively, corresponds to humanized antibody clone A6-3.

In one example, the effective amount of the antibody or antigen-binding fragment thereof for treating stroke or for use in the manufacture of a medicament for treating stroke is from 0.1 mg/kg to 15 mg/kg, or from 0.2 mg/kg to 14 mg/kg, or from 0.4 mg/kg to 13 mg/kg, or from 0.6 mg/kg to 12 mg/kg, or from 0.8 mg/kg to 11 mg/kg, or from 1 mg/kg to 10 mg/kg, or from 2 mg/kg to 9 mg/kg, or from 3 mg/kg to 8 mg/kg, or from 4 mg/kg to 7 mg/kg, or about 0.1 mg/kg, or about 0.2 mg/kg, or about 0.4 mg/kg, or about 0.6 mg/kg, or about 0.8 mg/kg, or about 1 mg/kg, or about 2 mg/kg, or about 3 mg/kg, or about 4 mg/kg, or about 5 mg/kg, or about 6 mg/kg, or about 7 mg/kg, or about 8 mg/kg, or about 9 mg/kg, or about 10 mg/kg, or about 11 mg/kg, or about 12 mg/kg, or about 13 mg/kg, or about 14 mg/kg, or about 15 mg/kg. Generally, an effective dosage may be in the range of about 0.1 mg to about 15 mg per kg body weight of the subject; about 0.2 mg to about 14 mg per kg body weight of the subject; about 0.4 mg to about 13 mg per kg body weight of the subject; about 0.6 mg to about 12 mg per kg body weight of the subject; about 0.8 mg to about 11 mg per kg body weight of the subject; or about 1 mg to about 10 mg per kg body weight of the subject; or about 2 mg to about 9 mg per kg body weight of the subject; about 3 mg to about 8 mg per kg body weight of the subject; about 4 mg to about 7 mg per kg body weight of the subject; or about 0.1 mg per kg body weight of the subject, or about 0.2 mg per kg body weight of the subject; or about 0.4 mg per kg body weight of the subject, or about 0.6 mg per kg body weight of the subject, or about 0.8 mg per kg body weight of the subject, or about 1 mg per kg body weight of the subject, or about 2 mg per kg body weight of the subject, or about 3 mg per kg body weight of the subject, or about 4 mg per kg body weight of the subject, or about 5 mg per kg body weight of the subject, or about 6 mg per kg body weight of the subject, or about 7 mg per kg body weight of the subject, or about 8 mg per kg body weight of the subject, or about 9 mg per kg body weight of the subject, or about 10 mg per kg body weight of the subject, or about 11 mg per kg body weight of the subject, or about 12 mg per kg body weight of the subject, or about 13 mg per kg body weight of the subject, or about 14 mg per kg body weight of the subject, or about 15 mg per kg body weight of the subject. In one example, the effective dosage is about 1 mg per kg body weight of the subject.

In one example, the effective amount of the antibody or antigen-binding fragment thereof for treating stroke or for use in the manufacture of a medicament for treating stroke is administered within about 0.5 hour, about 1.0 hour, about 1.5 hours, about 2.0 hours, about 2.5 hours, about 3.0 hours, about 3.5 hours, about 4.0 hours, about 4.5 hours, about 5.0 hours, about 5.5 hours, about 6.0 hours, or about 6.5 hours, or about 6.5 hours or more after the onset of the first stroke symptom(s). In one example, the effective amount of the antibody or antigen-binding fragment thereof for treating stroke or for use in the manufacture of a medicament for treating stroke is administered about 6.5 hours or more after the onset of the first stroke symptom(s). In one example, the effective amount of the antibody or antigen-binding fragment thereof for treating stroke or for use in the manufacture of a medicament for treating stroke is administered within about 3.0 hours or within about 6.0 hours after the onset of the first stroke symptom(s). In one example, the effective amount of the antibody or antigen-binding fragment thereof for treating stroke or for use in the manufacture of a medicament for treating stroke is administered within about 3.0 hours after the onset of the first stroke symptom(s). In one example, the effective amount of the antibody or antigen-binding fragment thereof for treating stroke or for use in the manufacture of a medicament for treating stroke is administered within about 6.0 hours after the onset of the first stroke symptom(s).

A stroke is a life-threatening medical condition that can occur due to a disruption in the blood supply to the brain. In one example, the stroke is selected from the group consisting of hemorrhagic stroke and ischemic stroke. In one example, the stroke is ischemic stroke.

In one example, the antibody or antigen-binding fragment thereof for treating stroke or for use in the manufacture of a medicament for treating stroke inhibits TRPM4 activity. In one example, the antibody or antigen-binding fragment thereof for treating stroke or for use in the manufacture of a medicament for treating stroke inhibits TRPM4 activity by inhibiting TRPM4 current. In one example, the antibody or antigen-binding fragment thereof for treating stroke or for use in the manufacture of a medicament for treating stroke inhibits TRPM4 activity by internalizing membrane TRPM4 protein. In one example, the antibody or antigen-binding fragment thereof for treating stroke or for use in the manufacture of a medicament for treating stroke inhibits TRPM4 activity by inhibiting TRPM4 current and internalizing membrane TRPM4 protein. In one example, the antibody or antigen-binding fragment thereof for treating stroke or for use in the manufacture of a medicament for treating stroke inhibits TRPM4 activity by internalizing membrane TRPM4 protein, which downregulates TRPM4 surface expression. In one example, under diseased conditions, inhibiting TRPM4 activity refers to blocking, preventing or disrupting the upregulated or activated protein function of TRPM4.

In one example, TRPM4 inhibition by the antibody or antigen-binding fragment thereof as disclosed herein improves vascular integrity, wherein the blood vessel(s) in a stroke subject treated with the antibody or antigen-binding fragment thereof as disclosed herein exhibit a longer vasculature and/or a larger diameter compared to an untreated stroke subject. A longer vasculature indicates less vascular damage, and a larger diameter suggests that more blood vessels are successfully recanalized. In an untreated stroke subject, the downstream vasculature of a damaged blood vessel usually has a smaller diameter as the blood vessels remain closed. In one example, the antibody or antigen-binding fragment thereof as disclosed herein increases vascular length and/or vascular diameter of affected blood vessel(s). In one example, with an improved vascular integrity due to the TRPM4 inhibition by the antibody or antigen-binding fragment thereof as disclosed herein, neuroinflammation is ameliorated or alleviated accordingly. In one example, with an improved vascular integrity due to the TRPM4 inhibition by the antibody or antigen-binding fragment thereof as disclosed herein, cerebral blood flow is improved or increased accordingly. In one example, under hypoxic condition, the antibody or antigen-binding fragment thereof as disclosed herein reduces oncotic cell death (such as in vascular endothelial cells), thereby improving and/or protecting vascular integrity. In one example, the antibody or antigen-binding fragment thereof as disclosed herein reduces infarct formation and/or infarct volume. In one example, the antibody or antigen-binding fragment thereof as disclosed herein inhibits neuronal cell swelling, such as hypoxia-induced neuronal cell swelling. In one example, the antibody or antigen-binding fragment thereof as disclosed herein reduces hypoxia-induced excitotoxicity in neurons after stroke, thus achieving neuroprotection. In one example, the antibody or antigen-binding fragment thereof as disclosed herein extends the therapeutic window for reperfusion. In one example, the current therapeutic window for reperfusion (such as by giving tPA) is about 4.5 hours after the onset of the first stroke symptom(s). In one example, the antibody or antigen-binding fragment thereof as disclosed herein extends the therapeutic window for reperfusion (such as by giving tPA) from about 4.5 hours to about 5.0 hours, about 5.5 hours, about 6.0 hours, about 6.5 hours, or about 6.5 hours or more after the onset of the first stroke symptom(s). In one example, the antibody or antigen-binding fragment thereof as disclosed herein extends the therapeutic window for reperfusion (such as by giving tPA) by about 10%, by about 20%, by about 30%, by about 40%, by about 50%, by about 60%, by about 70%, by about 80%, by about 90%, or by about 100%. Clinically, it means that stroke patients will have more time to receive the reperfusion therapy (such as tPA), and patients with early reperfusion tend to achieve a better outcome. In one example, the antibody or antigen-binding fragment thereof as disclosed herein extends the therapeutic window for reperfusion (such as by giving tPA) from about 4.5 hours to about 6.0 hours after the onset of the first stroke symptom(s). In one example, the antibody or antigen-binding fragment thereof as disclosed herein reduces or prevents reperfusion injury. In one example, the antibody or antigen-binding fragment thereof as disclosed herein is beneficial and may be used for both early and delayed reperfusion, thereby helping to reduce reperfusion injury. In one example, the antibody or antigen-binding fragment thereof as disclosed herein is beneficial and may be used for the time period between the early and delayed reperfusion, thereby helping to reduce reperfusion injury. In one example, early reperfusion means reperfusion therapy (such as the antibody or antigen-binding fragment thereof as disclosed herein and/or tPA) is given less than or within about 3 hours after the onset of the first stroke symptom(s). In one example, delayed reperfusion means reperfusion therapy (such as the antibody or antigen-binding fragment thereof as disclosed herein and/or tPA) is given more than about 4.5 hours after the onset of the first stroke symptom(s). In one example, delayed reperfusion means reperfusion therapy (such as the antibody or antigen-binding fragment thereof as disclosed herein and/or tPA) is given about 6.0 hours after the onset of the first stroke symptom(s). In one example, the effective amount of the antibody or antigen-binding fragment thereof treats stroke by increasing vascular length and/or vascular diameter of affected blood vessel(s), preventing vascular damage, recanalizing blood vessels, reducing infarct formation and/or infarct volume, inhibiting neuronal cell swelling, extending the therapeutic time window for reperfusion, reducing or preventing stroke reperfusion injury, alleviating neuroinflammation and/or reducing hypoxia-induced excitotoxicity in neurons.

In one example, the effective amount of the antibody or antigen-binding fragment thereof treats stroke by reducing one or more symptoms of stroke in the subject, wherein optionally the symptom of stroke is selected from the group consisting of confusion, severed numbness or weakness to one side or part of the body, severe headache, vision impairment, dizziness, walking difficulties, loss of balance or coordination, and slurred speech.

In one example, the antibody or antigen-binding fragment thereof as disclosed herein may be used in or administered to a stroke patient who is ineligible for reperfusion therapy. In one example, the reperfusion therapy includes thrombolysis and/or thrombectomy. In one example, there is no limit on the time to use or administer the antibody or antigen-binding fragment thereof as disclosed herein to a stroke patient who is ineligible for reperfusion therapy. In one example, the antibody or antigen-binding fragment thereof as disclosed herein may be used in or administered to a stroke patient who is ineligible for reperfusion therapy any time after the onset of the first stroke symptom(s). In one example, the antibody or antigen-binding fragment thereof as disclosed herein may be used in or administered to a stroke patient independently of a reperfusion therapy.

In one example, the method of treating stroke as disclosed herein further comprises administering to the subject one or more interventions selected from the group consisting of: (a) one or more surgical inventions; and (b) one or more therapeutic agents. In one example, the method of treating stroke as disclosed herein further comprises administering to the subject one or more surgical interventions. In one example, the method of treating stroke as disclosed herein further comprises administering to the subject one or more therapeutic agents. In one example, the method of treating stroke as disclosed herein further comprises administering to the subject one or more surgical interventions and one or more therapeutic agents. In one example of the use disclosed herein, the subject is to be further administered one or more interventions selected from the group consisting of: (a) one or more surgical inventions; and (b) one or more therapeutic agents. In one example of the use disclosed herein, the subject is to be further administered one or more surgical interventions. In one example of the use disclosed herein, the subject is to be further administered one or more therapeutic agents. In one example of the use disclosed herein, the subject is to be further administered one or more surgical interventions and one or more therapeutic agents. The surgical intervention may include any surgical interventions known in the art for treating stroke, such as, but is not limited to, thrombectomy, carotid endarterectomy, and craniotomy. The therapeutic agent for further administration in the treatment of stroke may include any agents, such as small molecule drug, antibody, oligonucleotide drug, etc. In one example, the therapeutic agent for further administration may include, but is not limited to, a thrombolytic agent, non-steroidal anti-inflammatory drugs, antiplatelets, and anticoagulants. In one example, the therapeutic agent for further administration is a thrombolytic agent. In one example, the thrombolytic agent is selected from the group consisting of tissue plasminogen activator (tPA), streptokinase, urokinase, and anistreplase. In one example, the thrombolytic agent is tPA.

In one example, the present disclosure refers to a kit for treating stroke comprising the antibody or antigen-binding fragment thereof disclosed herein and/or instructions for use. In some examples, the reagents provided in the kit as described herein may be provided in separate containers comprising the components independently distributed in one or more containers.

As used in this application, the singular form “a,” “an,” and “the” include plural references unless the context clearly dictates otherwise.

As used herein, the term “at least” when used in the context of a value is understood to mean “greater than or equal to” said value. For example, a sequence having “at least 85% sequence identity” to another sequence would be understood to include a sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the other sequence. In another example, an antibody or antigen-binding fragment thereof comprising a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11 would be understood to include an antibody or antigen-binding fragment thereof comprising a heavy chain variable region of SEQ ID NO: 7 (i.e. 100% sequence identity), and a light chain variable region of SEQ ID NO: 11 (i.e. 100% sequence identity).

The term “nucleic acid” refers to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides.

The term “antibody” means an immunoglobulin molecule able to bind to a specific epitope on an antigen. Antibodies can be comprised of a polyclonal mixture, or may be monoclonal in nature. Further, antibodies can be entire immunoglobulins derived from natural sources, or from recombinant sources. The antibodies of the present disclosure may exist in a variety of forms, including for example as a whole antibody, or as an antibody fragment, or other immunologically active fragment thereof, such as complementarity determining regions. Similarly, the antibody may exist as an antibody fragment having functional antigen-binding domains, that is, heavy and light chain variable domains. Also, the antibody fragment may exist in a form selected from the group consisting of, but not limited to: Fv, Fab, F (ab) 2, scFv (single chain Fv), dAb (single domain antibody), bi-specific antibodies, diabodies and triabodies As used in this application, “antibody fragment” refers to an antibody which has been reduced in length by one or more amino acids while retaining its antigenic activity.

The term “humanized antibody” refers to antibodies from non-human species whose protein sequences have been modified to increase their similarity to antibody variants produced naturally in humans.

The term “antibody variant” as used herein refers to antibodies with differences in their amino acid sequences but still retain their binding specificities to their antigens. For example, one or more conservative amino acid substitutions may be made to the TRPM4 antibody or its antigen-binding fragment thereof while retaining binding specificity to TRPM4.

As used herein “sequence identity” refers to the residues in two sequences that are the same when aligned for maximum correspondence over a specified window of comparison by means of computer programs known in the art such as GAP provided in the GCG program package (Program Manual for the Wisconsin Package, Version 11, 2005, Genetics Computer Group, 575 Science Drive, Madison, Wisconsin, USA 53711) and DNAstar Lasergene.

As used herein the term “treatment” refers to any and all uses which remedy a disease state or symptoms, prevent the establishment of disease, or otherwise prevent, hinder, retard, or reverse the progression of disease or other undesirable symptoms in any way whatsoever.

In the context of this specification, the term “an effective amount” includes within its meaning a non-toxic but sufficient amount of an agent (for example, the antibody or its antigen-binding fragment thereof of the present disclosure) to provide the desired effect. The exact amount required will vary from subject to subject depending on factors such as the species being treated, the age and general condition of the subject, the particular agent (for example, the antibody or its antigen-binding fragment thereof of the present disclosure) being administered and the mode of administration and so forth. Thus, it is not possible to specify an exact “effective amount”. However, for any given case, an appropriate “effective amount” may be determined by one of skill in the art using appropriate means.

In one example, the antibody or its antigen-binding fragment thereof may be administered to a subject in need thereof. Convenient modes of administration include injection (subcutaneous, intravenous, etc.), oral administration, inhalation, transdermal application, topical creams or gels or powders, or rectal administration. Depending on the route of administration, the antibody or its antigen-binding fragment thereof may be coated with a material to protect it from the action of enzymes, acids and other natural conditions which may inactivate its therapeutic activity. The antibody or its antigen-binding fragment thereof may also be administered parenterally or intraperitoneally.

Dispersions of the antibody or its antigen-binding fragment thereof according to the present disclosure may also be prepared in glycerol, liquid polyethylene glycols, and mixtures thereof and in oils. Under ordinary conditions of storage and use, pharmaceutical preparations may contain a preservative to prevent the growth of microorganisms.

The term “pharmaceutical composition” refers to a preparation containing a pharmaceutically active ingredient which is suitable for administration to a subject. The term “pharmaceutically acceptable carrier” refers to an ingredient in a pharmaceutical composition, other than the pharmaceutically active ingredient, which is nontoxic to a subject. The use of such “pharmaceutically acceptable carrier” is well known in the art. A pharmaceutically acceptable carrier includes, but is not limited to, a buffer, excipient, stabilizer, or preservative. The antibody or its antigen-binding fragment thereof may be formulated for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

In one embodiment, the antibody or its antigen-binding fragment thereof may be administered by injection. In the case of injectable solutions, the carrier can be a solvent or dispersion medium containing, for example, water, ethanol, polyol (for example, glycerol, propylene glycol, and liquid polyetheylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by including various anti-bacterial and/or anti-fungal agents. Suitable agents are well known to those skilled in the art and include, for example, parabens, chlorobutanol, phenol, benzyl alcohol, ascorbic acid, thimerosal, and the like. In many cases, isotonic agents, for example, sugars, polyalcohols such as mannitol, sorbitol, sodium chloride may be included in the pharmaceutical composition. Prolonged absorption of the injectable pharmaceutical compositions can be brought about by including in the pharmaceutical composition an agent which delays absorption, for example, aluminium monostearate and gelatin.

Sterile injectable solutions can be prepared by incorporating the analogue in the required amount in an appropriate solvent with one or a combination of ingredients enumerated above, as required, followed by filtered sterilisation. Generally, dispersions are prepared by incorporating the analogue into a sterile vehicle which contains a basic dispersion medium and the required other ingredients from those enumerated above.

Single or multiple administrations of the pharmaceutical compositions according to the present disclosure may be carried out. One skilled in the art would be able, by appropriate means, to determine effective, non-toxic dosage levels of the antibody or its antigen-binding fragment thereof and/or pharmaceutical composition of the present disclosure and an administration pattern which would be suitable for treating the diseases and/or infections to which the antibody or its antigen-binding fragment thereof and pharmaceutical compositions are applicable.

Further, it will be apparent to one of ordinary skill in the art that the optimal course of treatment, such as the number of doses of the antibody or its antigen-binding fragment thereof or pharmaceutical composition of the present disclosure given per day for a defined number of days, can be ascertained using convention course of treatment determination tests.

As used herein, the term “increase” refers to a rise in amount, expression level or number on a positive scale. Conversely, the term “decrease” indicates a change on a negative scale.

As used herein, the term “about” in the context of concentration of a substance, size of a substance, length of time, or other stated values means +/−5% of the stated value, or +/−4% of the stated value, or +/−3% of the stated value, or +/−2% of the stated value, or +/−1% of the stated value, or +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The present disclosure illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the present disclosure embodied therein herein disclosed may be resorted to by those skilled in the art, and that such modifications and variations are considered to be within the scope of this present disclosure.

The disclosure has been described broadly and generically herein. Each of the narrower species and subgeneric groupings falling within the generic disclosure also form part of the present disclosure. This includes the generic description of the present disclosure with a proviso or negative limitation removing any subject matter from the genus, regardless of whether or not the excised material is specifically recited herein.

Other embodiments are within the following claims and non-limiting examples.

### Examples

### Methods

### Antibody Humanization by CDR Grafting Plus Back Mutation

The structure of parental antibody was modelled by computer-aided homology modelling program. Humanized antibodies were designed using CDR grafting. Briefly, the CDRs of parental antibody were grafted into the human acceptors to obtain humanized light chains and humanized heavy chains for each parental antibody. 4 heavy chains (VH1, VH2, VH3 and VH4) and 4 light chains (VL1, VL2, VL3 and VL4) were paired with each other for affinity ranking experiment.

### Production of Chimeric and Humanized Antibodies

The DNA sequences encoding the chimeric and humanized antibodies heavy and light chains were synthesized and inserted into pcDNA3.4 vector to construct expression plasmids of full-length IgGs. The designed plasmids of heavy and light chain were sent for transfection to HEK 293 or CHO cells using Lipofectamine 2000. The culture media for both HEK 293 and CHO cells was: Dulbecco's Modified Eagle's Medium (DMEM) (12800017; Thermo Fisher Scientific, USA) supplemented with 10% fetal bovine serum (10500064, Thermo scientific); 1.74 g/L sodium bicarbonate; 1.2 g/L HEPES; and 100 U/mL Penicilin-Streptomycin (15140122; thermo scientific). The recombinant IgGs secreted to the medium were purified using protein. The purified antibody was buffer-exchanged into PBS using PD-10 desalting column. The concentration and purity of the purified protein were determined by OD280 and SDS-PAGE, respectively.

### Affinity Ranking of Chimeric and Humanized Antibodies

For affinity ranking, antibodies were captured on the sensor chip through Fc capture method. Peptide-biotin was used as the analyte. The surface was regenerated before the injection of another antibody. The process was repeated until all antibodies were analyzed. The off-rates of antibodies were obtained from fitting the experimental data locally to 1:1 interaction model using the Biacore 8K evaluation software. The antibodies were ranked by their dissociation rate constants (off-rates, kd). Based on the ranking result, the top 3 clones were selected.

### Affinity Measurement of Purified Humanized IgGs

The affinity of purified antibody binding to Peptide-biotin was individually determined using a Surface Plasmon Resonance (SPR) biosensor, Biacore 8K (GE Healthcare). Antibodies were captured on the sensor chip through Fc capture method. Peptide-biotin was used as the analyte. The data of dissociation (kd) and association (ka) rate constants were obtained using Biacore 8K evaluation software. The equilibrium dissociation constants (KD) were calculated from the ratio of kd over ka.

### Generation of TRPM4 Homozygous and Heterozygous Rats

The humanised TRPM4 rat model (TRPM4 heterozygous/homozygous rats) was produced using the In-Fusion cloning technology. The TRPM4 heterozygous rats were obtained by mating homozygous rats with wild-type rats. The rat TRPM4 gene (GenBank accession number: NM_001136229.1; Ensembl: ENSRNOG00000020714) is located on rat chromosome 1. Rat TRPM4 contains 25 exons and Exon 1 was selected as target site for CRISPR/Cas-mediated genome engineering. Following off-target analysis of potential gRNAs, one gRNA with the sequence of AGAGCAGGTATCGCACAGCGCGG was selected. The gRNAs targeting vectors were constructed and confirmed by sequencing. The donor vector which is flanked by homologous arms was constructed too, containing the rat TRPM4 coding sequences (CDS) in which the antibody binding sequence QDRSLPSILRRVFYRPYLQIFGQIPQEEMDVALMNPSNCSAERGSWAHPEGPV was human sequence replaced by the corresponding RDSDFPSILRRVFYRPYLQIFGQIPQEDMDVALMEHSNCSSEPGFWAHPPGAQ. After confirming with DNA sequencing, Cas9 mRNA and gRNA generated by in vitro transcription were co-injected into fertilised eggs with donor vector for knock-in (KI) rat production using Sprague-Dawley (SD) rats. The pups were genotyped using polymerase chain reaction (PCR) followed by sequence analysis.

### Middle Cerebral Artery Occlusion (MCAO) Protocol

Male transgenic Wistar rats carrying human TRPM4 sequences weighing approximately 250-280 g were anesthetised with ketamine (75 mg/kg) and xylazine (10 mg/kg) intraperitoneally. Relative regional cerebral blood flow of the animals was monitored by a Laser-Doppler flowmetry. Heart rate, blood pressure, and rectal temperature were monitored using a data acquisition system. The body temperature was maintained at 37° C.+0.5° C. with a warm pad throughout the procedure. The left common carotid artery (CCA), internal carotid artery (ICA), and external carotid artery (ECA) were dissected out. A silicon-coated filament was introduced into the left ICA through ECA. Cerebral blood flow of the animals was monitored by a Laser-Doppler flowmetry. Animals with ≤70% cerebral blood flow reduction were excluded from the study. Reperfusion was achieved by removing the filament gently from the ECA at 3 h following occlusion to establish a stroke reperfusion model. To establish a permanent stroke model, the filament was left inside the left ICA instead of removing the filament. This represents the condition in ischemic stroke patients without reperfusion. A single dose of M4H antibody or control human IgG at a dose of 1 mg/kg was injected intravenously via tail vein at 2 h after occlusion (1 h before recanalization).

### Immunostaining

HEK293 cells expressing human TRPM4 channel tagged with myc were used. The live cells were incubated with 20.8 μg/mL of the TRPM4 humanized antibodies or control human IgG for 40 mins at 37° C. The cells were fixed with 4% (v/v) paraformaldehyde (PFA) for 20 minutes at room temperature followed by permeabilization with 0.1% (v/v) Triton X-100 for 15 mins. The cells were then incubated 5% fetal bovine serum (FBS) for 30 mins and then incubated with the appropriate Alexa Fluor® 594 secondary antibody prepared in 5% FBS/PBS for 1 hour at room temperature to detect TRPM4 or control IgG. After which, the cells were added with the corresponding Alexa Fluor® 594 secondary antibody prepared in 5% FBS/PBS for 1 hour. The cells were counterstained with DAPI for 5 mins. Wheat Germ Agglutinin (WGA) Alexa Fluor 488 was used to stain plasma membrane. The cells were washed with PBS between incubations and before mounting on glass slides using the FluorSave™ reagent (Merck, NJ, USA). Fluorescent images were captured with a laser scanning confocal microscope system.

### 2,3,4-Triphenyltetrazolium Chloride (TTC) Staining and Infarct Volume Measurement

Twenty-four hours after surgery, the animals were sacrificed, and the brains were collected with cerebellum and overlying membranes being removed. The brains were sectioned into 8 slices using a brain-sectioning block, each with 2 min thickness. The brain slices were incubated for 30 min in a 0.1% solution of 2,3,4-triphenyltetrazolium chloride (TTC) at 37° C. The sections were scanned, and the infarct size was analysed using an image J analyser system with oedema-corrected.

### Electrophysiology in Cells and Dose-Dependent Inhibition of Humanized Antibodies

Whole-cell patch clamp was used to characterize the inhibitory effect of the humanized antibodies in HEK293 cells expressing human TRPM4 (Myc-DDK-tagged). Whole-cell currents were recorded at room temperature using a patch clamp amplifier (Multiclamp 700B equipped with Digidata 1440A, Molecular Devices, CA, USA). Patch electrodes were pulled using a Flaming/Brown micropipette puller (P-1000, Sutter Instrument, CA, USA) and polished with a microforge (MF200, World Precision Instruments Inc. FL, USA). The bath solution contained (in millimole/litre) NaCl 140, CaCl2 2, KCl 2, MgCl2 1, glucose 20, and HEPES 20 at pH 7.4. The internal solution contained (in millimole/litre) CsCl 156, MgCl2 1, EGTA 10, and HEPES 10 at pH 7.2 adjusted with CsOH. Additional Ca2+ was added in the pipette solution to get 7.4 μM free Ca2+, calculated using WEBMAXC v2.10. Humanized antibodies or control human IgG was added into bath solution 30 min before recording at a concentration of 20.8 μg/mL. The current-voltage relations were measured by applying voltage ramps for 250 ms from −80 to +80 mV at a holding potential of 0 mV. The sampling rate was 20 kHz and the filter setting was 1 kHz. Data were analysed using pClamp10, version10.2 (Molecular Devices, CA, USA). Hypoxia was induced by applying a bath solution containing 5 mM NaN3 and 10 mM 2-deoxyglucose (2-DG) continuously through a MicroFil (34 Gauge, World Precision Instruments Inc. USA) around 10 μm away from the recording cells. The flow rate was set at 200 μL/min. For dose-response curve, the HEK293 cells were incubated with various doses of humanized antibody clone A6 (M4H). The currents before and after hypoxia induction were recorded and compared to calculate IC₅₀.

### Rotarod Test

Motor function was evaluated using a rotarod apparatus (Ugo Basile, Gemonio, Italy). The impairment of motor function was quantified by observing the latency with which the rats fell off the apparatus. Before stroke induction, the rats received three training trials with 15-min intervals each day for five consecutive days. The rotarod was set to accelerate from 4 to 80 rpm within 10 min. One day before operation, the mean duration of time that the animals remained on the device was recorded as an internal baseline control. After surgery, the mean duration of latency was recorded at different time points for comparison among different treatments.

### Neurological Assessment

The Bederson scale, which is a global neurological assessment, was used to measure neurological deficit in the rats. The assessment included forelimb flexion, resistance to lateral push and circling behaviour. For each test, the rats were graded on a scale of 0-3 for evaluating the global neurological severity. The Bederson scale was obtained at different time points post-MCAO for comparison.

### Antibody-Dependent Cell-Mediated Cytotoxicity (ADCC) Assay

Human Lung Microvascular Endothelial cells (HULEC-5a) and Human Umbilical Vein Endothelial cells (HUVEC) cells were harvested and used as target cells by resuspending them in assay buffer (RPMI1640 medium+10% FBS). The target cell density adjusted before the target cell suspension was transferred to the assay plate (20,000 cells/well, 50 μL/well). The assay plate was then incubated in the cell incubator (at 37° C. with 5% CO2) overnight. After overnight incubation, 4× sample working solution was added (at 25 μL/well) to the target cell suspension in the assay plate. Effector cells (such as monocyte or macrophages) were also harvested and resuspended in assay buffer (RPMI1640 medium+10% FBS). Effector cell density was adjusted before the effector cell suspension was transferred to the assay plate (containing the target cell suspension) according to the plate map schemes (120,000 cells/well, 25 μL/well). The assay plate was then incubated in the cell incubator (at 37° C. with 5% CO2) for 6 hours. After incubation, Bio-Lite Luciferase working solution (100 μL/well) was added to corresponding wells in the assay plate. The luminescence signal was read with PHERAstar. Dose-response curves were then plotted with the relative luminescence unit against the sample concentration. Relative EC50 values were obtained using four-parameter function as follows, characterizing sigmoid curve where luminescence value is against the concentration of the test samples:

$$Y = Bottom + ( Top - Bottom ) / ( 1 + 10 \hat{} ( ( Log EC 50 - X ) * HillSlope ) )$$

$$X = Log ( Concentration ) ; Y = Luminescence values .$$

### Results

### Effects of TRPM4-Blocking Antibody in Stroke

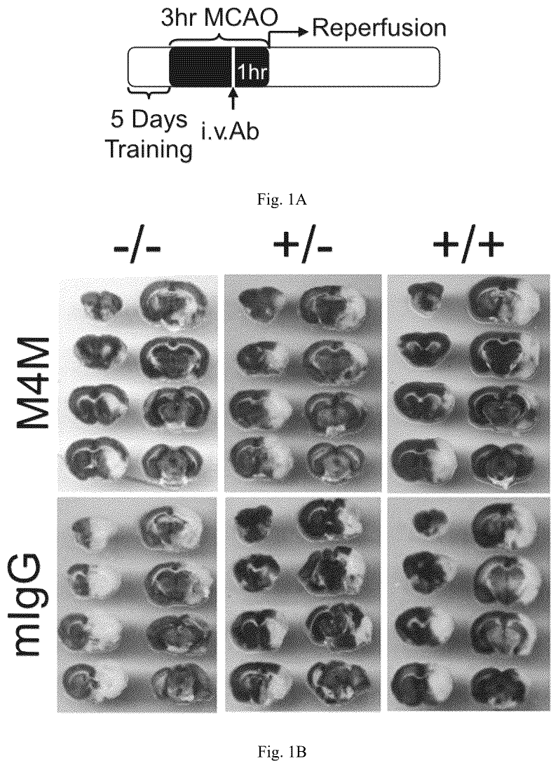

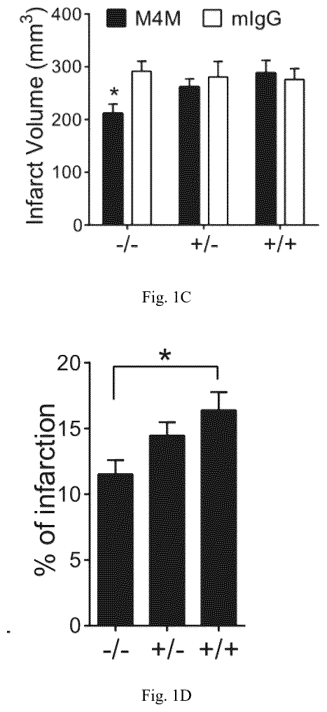

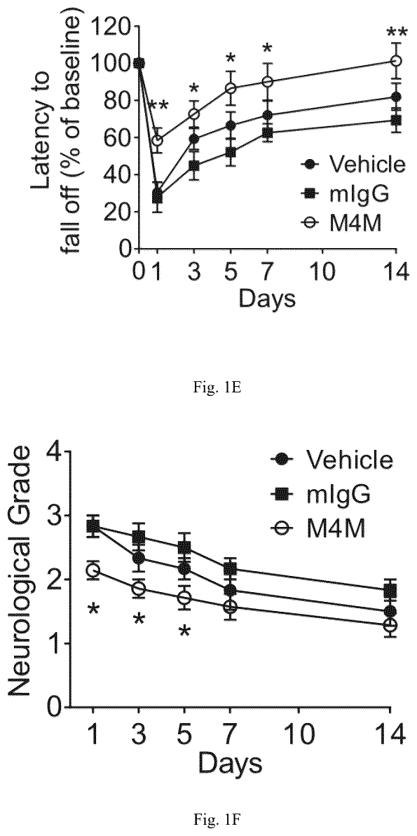

To examine the in vivo effect of M4M in stroke, an ischemia reperfusion model was established in rats in which the middle cerebral arteries were transiently occluded for 3 hrs followed by recanalization. M4M or control mouse IgG (mIgG) was applied intravenously 1 hr before recanalization (FIG. 1A). This experimental design mimicked the clinical scenario in which the antibody can be delivered together with the reperfusion drug tPA. Infarct formation was assessed 1 day after operation in wild-type, heterozygous, and homozygous rats to compare the effect of M4M with mIgG (FIG. 1B). A summary of infarct volume shows that compared to mIgG, M4M treatment significantly reduced infarct formation in homozygous rats, but not in heterozygous or wild-type rats (FIG. 1C). The absence of therapeutic effect of M4M in wild-type rats is because M4M binds to human TRPM4 but not rodent TRPM4. When the infarct volume of M4M treatment in all three groups of animals (FIG. 1D) was compared, the infarct volume in the homozygous group was significantly lower than the infarct volume in the wild-type group. The average value of the infarct volume in the heterozygous group was higher than that of the homozygous group, and lower than the wild-type group. However, there was no significance difference between the heterozygous group and either group.

To verify that the infarct volume reduction by M4M in homozygous rats was associated with functional improvement, the Rotarod test was performed to assess the motor functions in homozygous rats (FIG. 1E). One day after operation, the performance of the M4M group was significantly better than that of the mIgG group or the vehicle group. On day 3, 5, 7, and 10, the M4M group demonstrated significantly better motor functions compared to the mIgG group, but not to the vehicle group. Neurological deficit was further examined using a neurological severity score system. The highest score was observed at day 1 after operation, and the score gradually decreased in the following days (FIG. 1F). Similar as the Rotarod test, the neurological scores of the M4M group were significantly lower than those of both mIgG and vehicle groups at day 1. At days 3 and 5, the M4M group was significantly lower than the mIgG group, but not the vehicle group. The lower value of the neurological scores in the M4M group in FIG. 1F implies that the M4M group demonstrated better neurological functions and less neurological impairment compared to the mIgG and vehicle groups.

### Validation of Antibody Binding Affinity of Humanized Antibodies Against TRPM4

Humanized antibodies were generated by replacing the framework regions (FRs) from mouse M4M antibody with counterparts from human, while maintaining the antibody's complementarity-determining regions (CDRs) binding regions. The antibody binding affinity of the chimeric and humanized antibodies was assessed by surface plasmon resonance (SPR) as shown in Table 1. Antibody clone A1 is the chimeric antibody and antibody clones A2-7 are humanized antibodies. Based on the results obtained from SPR, humanized clone A2 has the strongest SPR signal. The antigen binding kinetics to the chimeric and humanized antibodies are shown in Table 2. For affinity ranking, antibodies were captured on the sensor chip through Fc capture method. Peptide-biotin was used as the analyte. The surface was regenerated before the injection of another antibody. The process was repeated until all antibodies are analyzed. The off-rates of antibodies were obtained from fitting the experimental data locally to 1:1 interaction model using the Biacore 8K evaluation software. The antibodies were ranked by their dissociation rate constants (off-rates, kd). Based on the ranking result, the top humanized clones were selected. Table 3 shows the humanization design and back mutation sites in the framework regions of the heavy and light chain regions of the humanized antibody clones. Bolded and underlined amino acids correspond to the back mutation sites in the framework regions.

Humanized antibody clones (A2-A7) were generated by combining various heavy and light chain variable domains (VH1-VH4 variable regions and VL1-VL4 variable regions). The amino acid sequence of the variable domains for heavy and light chains of the humanized antibodies are as follows:

VH1 variable region:
(SEQ ID NO: 7)
QVQLQESGPGLVKPSQTLSLTCTVSGYSITSDYAWNWIRQHPGKGLEWI
GYLSYSGVTSYNPSLKGRVTISVDTSKNQFSLKLSSVTAADTAVYYCAR
KGTYYRYEGSYWYFDVWGQGTTVTVSS
VH2 variable region:
(SEQ ID NO: 8)
QVQLQESGPGLVKPSQTLSLTCTVSGYSITSDYAWNWIRQHPGKGLEWM
GYLSYSGVTSYNPSLKGRVTISRDTSKNQFSLKLSSVTAADTAVYYCAR
KGTYYRYEGSYWYFDVWGQGTTVTVSS
VL1 variable region:
(SEQ ID NO: 11)
DIQMTQSPSSLSASVGDRVTITCHANQNIDVWLSWYQQKPGKAPKLLIY
KTSNLHTGVPSRFSGSGSGTDFTFTISSLQPEDIATYYCQQGQSYPLTF
GGGTKLEIK
VL2 variable region:
(SEQ ID NO: 12)
DIQMTQSPSSLSASVGDRVTITCHANQNIDVWLSWYQQKPGKAPKLLIY
KTSNLHTGVPSRFSGSGSGTDFTLTISSLQPEDIATYYCQQGQSYPLTF
GGGTKLEIK
VL3 variable region:
(SEQ ID NO: 13)
DIQMTQSPSSLSASVGDRVTITCHANQNIDVWLSWYQQKPGNAPKLLIY
KTSNLHTGVPSRFSGSGSGTDFTFTISSLQPEDIATYYCQQGQSYPLTF
GGGTKLEIK
VL4 variable region:
(SEQ ID NO: 14)
DIQMTQSPSSLSASVGDRVTITCHANQNIDVWLSWYQQKPGNAPKLLIY
KTSNLHTGVPSRFSGSGSGTDFTLTISSLQPEDIATYYCQQGQSYPLTF
GGGTKLEIK

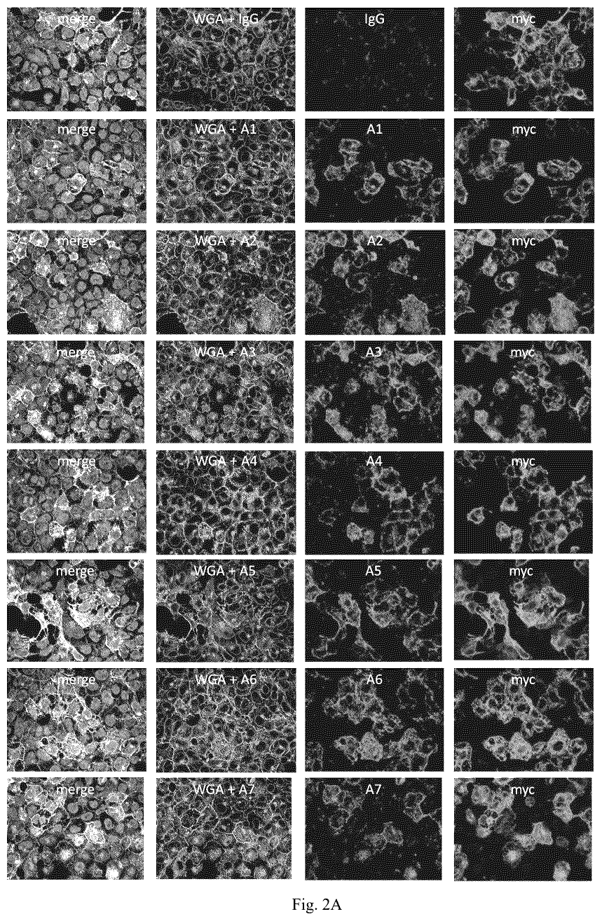

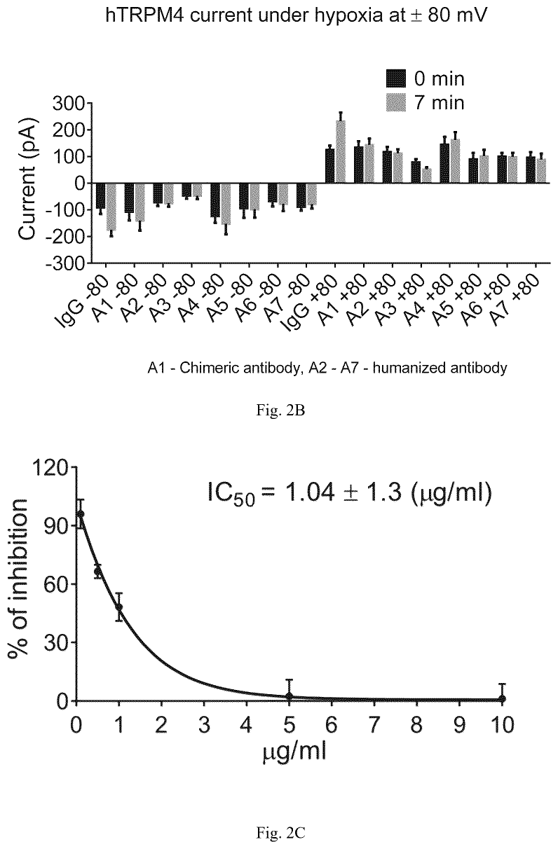

Biochemical and functional characterization were performed to evaluate the antigenic properties of the humanized antibodies. Immunofluorescent staining showed that all six humanized antibodies A2-7 and the chimeric antibody A1 could stain human TRPM4 in transfected HEK 293 cells (FIG. 2A). In addition to staining cytosolic TRPM4, colocalization with surface marker WGA suggested that these antibodies also recognized surface TRPM4. Next, functions of the humanized antibodies were evaluated by electrophysiology (FIGS. 2B and 2C). Human TRPM4 current was significantly elevated by a 7-minute hypoxia treatment in control IgG treated cells (FIG. 2B). All 6 humanized antibodies (A2-A7) and the chimeric antibody A1 successfully inhibited hypoxia-induced current increase at a concentration of 20 μg/ml. Since humanized antibody A2 has the least number of back mutation sites while maintaining excellent binding affinity, the dose-dependent effect of A2 on hypoxia-induced TRPM4 current increase was further determined.

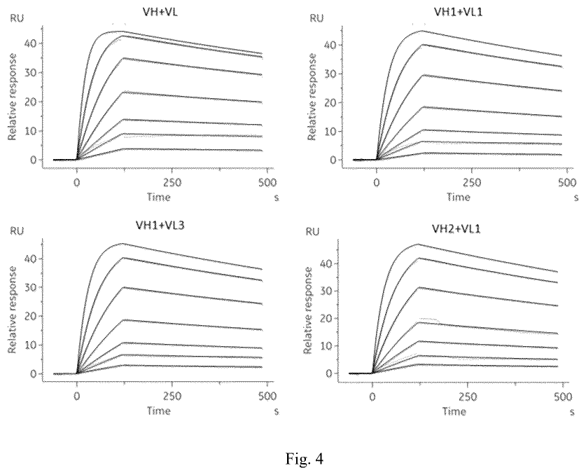

Using human IgG as a control, the IC₅₀ of A2 was determined to be 1.23 μg/ml in a dose-dependent inhibition assay, which was higher than the IC₅₀ of A6 (1.04 μg/ml). No difference was found when the dose was increased above 5 μg/ml (FIG. 2C). In addition, humanized antibody A6 demonstrated better performance in terms of drug stability. Therefore, humanized antibody A6 was selected for further animal study. The binding affinity and kinetics of different humanized antibodies (A1, A2, A4 and A6) to Peptide-biotin were also shown in the form of sensor-grams (FIG. 4). The overlaid smooth black lines of the sensor-grams represent the kinetic fit of the binding response signals at different antibody concentrations to a 1:1 interaction model.

TABLE 1
Binding kinetics of antibody to antigen

| Ligand | Analyte | Chi² (RU²) | ka (1/Ms) | kd (1/s) | KD (M) | Rmax (RU) |
|---|---|---|---|---|---|---|
| NC | peptide-biotin | NA | NA | NA | NA | NA |
| VH + VL | peptide-biotin | 1.61E−02 | 3.25E+05 | 1.50E−03 | 4.61E−09 | 24.3 |
| (A1) | | | | | | |
| VH1 + VL1 | peptide-biotin | 2.24E−02 | 2.49E+05 | 1.66E−03 | 6.66E−09 | 28.2 |
| (A2) | | | | | | |
| VH1 + VL2 | peptide-biotin | 2.66E−01 | 3.27E+05 | 3.21E−03 | 9.81E−09 | 25.4 |
| (A3) | | | | | | |
| VH1 + VL3 | peptide-biotin | 4.42E−02 | 2.52E+05 | 1.77E−03 | 7.00E−09 | 26.9 |
| (A4) | | | | | | |
| VH1 + VL4 | peptide-biotin | 8.68E−02 | 2.17E+05 | 2.17E−03 | 9.96E−09 | 24 |
| (A5) | | | | | | |
| VH2 + VL1 | peptide-biotin | 5.72E−02 | 1.84E+05 | 1.95E−03 | 1.06E−08 | 31.1 |
| (A6) | | | | | | |
| VH2 + VL2 | peptide-biotin | 9.44E−02 | 2.56E+05 | 2.95E−03 | 1.15E−08 | 29.6 |
| (A7) | | | | | | |
| VH2 + VL3 | peptide-biotin | 3.32E−02 | 2.03E+05 | 2.19E−03 | 1.08E−08 | 26.7 |
| VH2 + VL4 | peptide-biotin | 2.49E−02 | 2.15E+05 | 1.85E−03 | 8.62E−09 | 29.2 |
| VH3 + VL1 | peptide-biotin | 3.00E−02 | 3.42E+05 | 1.49E−03 | 4.36E−09 | 28.2 |
| VH3 + VL2 | peptide-biotin | 7.68E−02 | 4.38E+05 | 1.98E−03 | 4.53E−09 | 26.3 |
| VH3 + VL3 | peptide-biotin | 7.77E−02 | 4.09E+05 | 1.80E−03 | 4.40E−09 | 29.5 |
| VH3 + VL4 | peptide-biotin | 1.42E−01 | 6.08E+05 | 1.35E−03 | 2.22E−09 | 26.8 |
| VH4 + VL1 | peptide-biotin | 2.76E−02 | 3.05E+05 | 1.36E−03 | 4.47E−09 | 32.7 |
| VH4 + VL2 | peptide-biotin | 7.55E−02 | 3.03E+05 | 1.32E−03 | 4.37E−09 | 30.3 |
| VH4 + VL3 | peptide-biotin | 3.93E−02 | 3.32E+05 | 1.43E−03 | 4.32E−09 | 28.3 |
| VH4 + VL4 | peptide-biotin | 2.15E−01 | 4.54E+05 | 2.01E−03 | 4.43E−09 | 54.4 |

TABLE 2
Binding kinetics of antigen to antibodies

| Ligand | Analyte | Chi² (RU²) | ka (1/Ms) | kd (1/s) | KD (M) | Rmax (RU) |
|---|---|---|---|---|---|---|
| VH + VL | peptide-biotin | 1.24E−01 | 3.12E+05 | 5.43E−04 | 1.74E−09 | 44.2 |
| (A1) | | | | | | |
| VH1 + VL1 | peptide-biotin | 1.04E−01 | 1.91E+05 | 6.05E−04 | 3.16E−09 | 45.8 |
| (A2) | | | | | | |
| VH1 + VL3 | peptide-biotin | 7.35E−02 | 1.95E+05 | 6.20E−04 | 3.18E−09 | 46.1 |
| (A4) | | | | | | |
| VH2 + VL1 | peptide-biotin | 1.62E−01 | 1.65E+05 | 6.62E−04 | 4.02E−09 | 48.3 |
| (A6) | | | | | | |

TABLE 3
Humanization design and back mutation sites

| | Chain | | | | | | | |
|---|---|---|---|---|---|---|---|---|
| Germline | Type | FR1 | CDR1 | FR2 | CDR2 | FR3 | CDRS3 | FR4 |
| | VH | [symbol] | [symbol] | [symbol] | [symbol] | [symbol] | [symbol] | [symbol] |
| [symbol] | VH1 | [symbol] | [symbol] | [symbol] | [symbol] | [symbol] | [symbol] | [symbol] |
| | VH2 | [symbol] | [symbol] | [symbol] | [symbol] | [symbol] | [symbol] | [symbol] |
| | VH3 | [symbol] | [symbol] | [symbol] | [symbol] | [symbol] | [symbol] | [symbol] |
| | VH4 | [symbol] | [symbol] | [symbol] | [symbol] | [symbol] | [symbol] | [symbol] |
| | VL | [symbol] | [symbol] | [symbol] | [symbol] | [symbol] | [symbol] | [symbol] |
| [symbol] | VL1 | [symbol] | [symbol] | [symbol] | [symbol] | [symbol] | [symbol] | [symbol] |
| | VL2 | [symbol] | [symbol] | [symbol] | [symbol] | [symbol] | [symbol] | [symbol] |
| | VL3 | [symbol] | [symbol] | [symbol] | [symbol] | [symbol] | [symbol] | [symbol] |
| | VL4 | [symbol] | [symbol] | [symbol] | [symbol] | [symbol] | [symbol] | [symbol] |

[symbol]

indicates data missing or illegible when filed

### Effect of Humanized Antibodies in Stroke

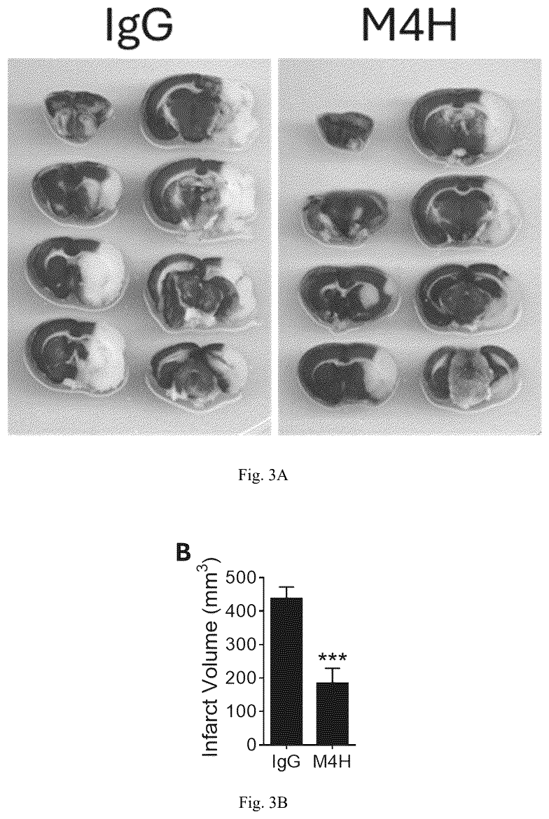

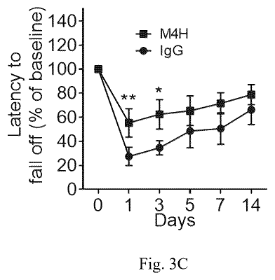

Humanized antibody A6 (renamed as M4H) was further examined in an animal model of stroke. The stroke reperfusion model was created by occluding the rat's left middle cerebral artery for 3 hours. Reperfusion was achieved by removing the filament. M4H or control human IgG at a dose of 1 mg/kg was administered 1 hour before recanalization, mimicking the clinical scenario of stroke reperfusion therapy. One day after treatment, staining of brains revealed that M4H treatment reduced infarct formation (FIG. 3A-B) compared to control human IgG treatment. Accordingly, motor functions of the rats as assessed by Rotarod showed improvement after M4H treatment (FIG. 3C). The results in FIGS. 3A-3C showed that humanized antibody M4H could reduce stroke reperfusion injury.

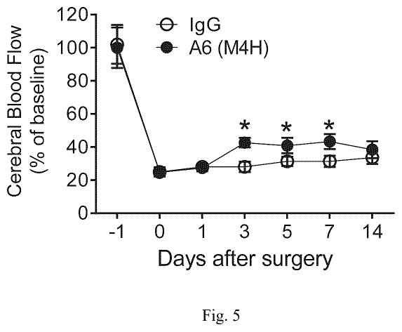

The effect of M4H was also further assessed in a permanent stroke model which was created by inserting a filament to occlude the rat's left middle cerebral artery. The filament was left inside the left middle cerebral artery to create the permanent stroke model. The permanent stroke model was generated to mimic an outcome of ischemic stroke whereby reperfusion therapy is no longer applicable. M4H or control human IgG at a dose of 1 mg/kg was injected intravenously at 2 h after performing the occlusion. Blood flow was monitored using a laser doppler machine at different time points after the surgery. Repeated ANOVA test was used to compare the difference in blood flow readings between M4H and control human IgG treatment. The results in FIG. 5 showed that application of M4H improved cerebral blood flow after day 3 of post-surgery.

### Cytotoxic Effects of Humanized Antibodies

M4H was generated with normal IgG1 which could initiate potential antibody-dependent cellular cytotoxicity & phagocytosis (ADCC & ADCP), and complement-dependent cytotoxicity (CDC). ADCC, ADCP and CDC are useful when the therapeutic antibodies are used to kill cancer cells. However, in stroke treatment, the aim is to protect neurons and vascular endothelial cells. To reduce potential ADCC, ADCP and/or CDC, additional variants A6-1, A6-2, and A6-3 were generated from the humanized antibody M4H. Humanized M4H antibody variants A6-1 and A6-2 were generated by introducing point mutation(s) in the IgG1 constant region of the heavy chain to reduce toxicity. A6-3 variant was generated by replacing the IgG1 constant region of the heavy chain with an IgG4 constant region containing point mutation(s) to reduce toxicity. These variants were then tested for ADCC activity against HULEC-5a and HUVEC cells lines which serve as target cells.

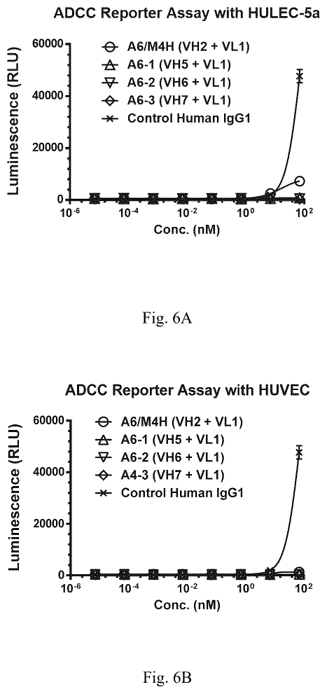

Control human IgG demonstrated high ADCC activity in both cell lines (FIGS. 6A and 6B). In HULEC-5a, although M4H (A6) exhibited higher ADCC activity than its variants A6-1, A6-2, and A6-3, the ADCC activity of M4H is still much lower when compared to control human IgG (FIG. 6A). Similar trends were found in HUVEC cells (FIG. 6B). Overall, variants A6-2 and A6-3 had the least ADCC activity, and therefore appeared to be the best options for therapy.

### DISCUSSION

The present disclosure describes humanized antibodies specific against TRPM4 and their use in treating stroke. The humanized antibodies disclosed herein effectively inhibits TRPM4 activity and has a therapeutic potential for stroke. The present disclosure has also shown the protective effect of TRPM4 specific blocking antibodies in stroke.

As discussed above, currently, the only potent treatment for acute ischemic stroke is reperfusion therapy. However, the reperfusion injury following recanalization of the blocked vessels limits the use of reperfusion therapy to a small portion of stroke patents. Therefore, there is an urgent need to develop novel treatment (such as humanized antibodies) to benefit more stroke patients.

The present disclosure describes for the first time the therapeutic use of humanized antibodies specific to TRPM4 in treating stroke. The humanized antibodies of the present disclosure have the following advantages:
1. If the humanized antibodies specific to TRPM4 is administered intravenously in combination (either simultaneously or sequentially) with reperfusion treatment such as tPA or thrombectomy, it can improve functional outcome in the stroke subject with less disabilities.
2. If a patient missed the 4.5 hours therapeutic time window for reperfusion treatment (such as tPA), and if the humanized antibodies specific to TRPM4 is administered after 4.5 hours but still within 6 hours after the onset of the first stroke symptom(s), the patient may still be able to receive reperfusion treatment (such as tPA).
3. The TRPM4-blocking humanized antibody can reduce reperfusion injury by protecting vasculature and improving neuronal survival. Reduction of reperfusion injury in patients receiving reperfusion therapy can reduce disabilities and mortality. Further, TRPM4-blocking antibody could potentially extend the therapeutic time window from 4.5 hours to 6 hours (as discussed in point 2 above) along with the amelioration of reperfusion injury. Thus, more stroke patients could benefit from the reperfusion therapy. The humanized antibodies of the present disclosure can serve as an adjuvant treatment together with reperfusion therapy such as tPA or thrombectomy.
4. Intravenous administration of the humanized antibodies specific to TRPM4 (independent of a reperfusion therapy) can also improve functional outcome in a stroke subject who is ineligible for reperfusion therapy, such as a subject who receives no reperfusion therapy.
5. The humanized antibodies specific to TRPM4 disclosed herein can be used safely as a therapeutic molecule, for example, the humanized antibodies exhibit low cytotoxic activities when used in treatment.

SEQUENCE LISTING

| SEQ ID | | |
|---|---|---|
| NO | Sequence Name | Sequence |
| 1 | heavy chain CDR1 | GYSITSDYAWN |
| 2 | heavy chain CDR2 | YLSYSGVTSYNPSLKG |
| 3 | heavy chain CDR3 | KGTYYRYEGSYWYFDV |
| 4 | Light chain CDR1 | HANQNIDVWLS |
| 5 | Light chain CDR2 | KTSNLHT |
| 6 | Light chain CDR3 | QQGQSYPLT |
| 7 | VH1 variable region | QVQLQESGPGLVKPSQTLSLTCTVSGYSITSDYAWNWIRQHPG |
| | | KGLEWIGYLSYSGVTSYNPSLKGRVTISVDTSKNQFSLKLSSVT |
| | | AADTAVYYCARKGTYYRYEGSYWYFDVWGQGTTVTVSS |
| 8 | VH2 variable region | QVQLQESGPGLVKPSQTLSLTCTVSGYSITSDYAWNWIRQHPG |
| | | KGLEWMGYLSYSGVTSYNPSLKGRVTISRDTSKNQFSLKLSSV |
| | | TAADTAVYYCARKGTYYRYEGSYWYFDVWGQGTTVTVSS |
| 9 | VH3 variable region | QVQLQESGPGLVKPSQTLSLTCTVSGYSITSDYAWNWIRQHPG |
| | | KGLQWMGYLSYSGVTSYNPSLKGRITISRDTSKNQFSLKLSSV |
| | | TAADTAVYYCARKGTYYRYEGSYWYFDVWGQGTTVTVSS |
| 10 | VH4 variable region | QVQLQESGPGLVKPSQTLSLTCTVSGYSITSDYAWNWIRQHPG |
| | | KGLQWMGYLSYSGVTSYNPSLKGRITISRDTSKNQFFLKLSSV |
| | | TAADTAVYYCARKGTYYRYEGSYWYFDVWGQGTTVTVSS |
| 11 | VLI variable region | DIQMTQSPSSLSASVGDRVTITCHANQNIDVWLSWYQQKPGKA |
| | | PKLLIYKTSNLHTGVPSRFSGSGSGTDFTFTISSLQPEDIATYYCQ |
| | | QGQSYPLTFGGGTKLEIK |
| 12 | VL2 variable region | DIQMTQSPSSLSASVGDRVTITCHANQNIDVWLSWYQQKPGKA |
| | | PKLLIYKTSNLHTGVPSRFSGSGSGTDFTLTISSLQPEDIATYYCQ |
| | | QGQSYPLTFGGGTKLEIK |
| 13 | VL3 variable region | DIQMTQSPSSLSASVGDRVTITCHANQNIDVWLSWYQQKPGNA |
| | | PKLLIYKTSNLHTGVPSRFSGSGSGTDFTFTISSLQPEDIATYYCQ |
| | | QGQSYPLTFGGGTKLEIK |
| 14 | VL4 variable region | DIQMTQSPSSLSASVGDRVTITCHANQNIDVWLSWYQQKPGNA |
| | | PKLLIYKTSNLHTGVPSRFSGSGSGTDFTLTISSLQPEDIATYYCQ |
| | | QGQSYPLTFGGGTKLEIK |
| 15 | TRPM4 Epitope | RDSDSNCSSEPGFWAHPPGAQ |
| 16 | TRPM4 Epitope | EPGF |
| 17 | Nucleotide sequence | ATGGGCTGGTCATGCATTATTCTGTTTCTGGTCGCAACTGCTAC |
| | encoding VH1 | AGGCGTGCATAGTCAAGTGCAGCTGCAGGAGAGCGGACCTGG |
| | | CCTGGTGAAGCCTTCTCAGACCCTGAGCCTCACCTGTACCGTG |
| | | TCCGGATATAGCATCACAAGCGACTACGCCTGGAATTGGATCA |
| | | GACAGCACCCAGGCAAGGGCCTGGAATGGATCGGCTACCTGTC |
| | | CTACAGCGGCGTGACATCTTATAACCCCAGCCTGAAAGGAAGA |
| | | GTGACCATCAGCGTGGACACCTCTAAGAACCAGTTCAGCCTGA |
| | | AGCTGTCTAGCGTCACAGCCGCTGATACCGCCGTGTACTACTGC |
| | | GCCAGAAAGGGCACCTACTACCGGTACGAGGGCAGCTACTGGT |
| | | ACTTCGACGTGTGGGGCCAGGGCACAACCGTGACCGTTTCCAG |
| | | CGCCAGCACCAAGGGCCCTTCCGTGTTTCCACTGGCCCCCTCCT |
| | | CTAAATCCACATCTGGCGGCACCGCCGCCCTGGGCTGTCTGGTG |
| | | AAGGACTACTTCCCAGAGCCTGTGACAGTGTCCTGGAACTCTGG |
| | | CGCCCTGACATCCGGCGTGCACACATTTCCAGCCGTGCTGCAGA |
| | | GCTCCGGCCTGTACAGCCTGTCTAGCGTGGTGACAGTGCCCTCC |
| | | TCTAGCCTGGGCACACAGACCTATATCTGCAACGTGAATCACAA |
| | | GCCAAGCAATACCAAGGTGGACAAGAAGGTGGAGCCCAAGTCC |
| | | TGTGATAAGACACACACCTGCCCCCCTTGTCCTGCTCCCGAGCT |
| | | GCTGGGCGGCCCTAGCGTGTTCCTGTTTCCACCCAAGCCTAAGG |
| | | ACACCCTGATGATCTCCCGGACACCCGAGGTGACCTGCGTGGT |
| | | GGTGGACGTGTCTCACGAGGATCCTGAGGTGAAGTTCAACTGG |
| | | TATGTGGATGGCGTGGAGGTGCACAATGCCAAGACCAAGCCCA |
| | | GAGAGGAGCAGTACAACTCTACATATAGGGTGGTGAGCGTGCT |
| | | GACCGTGCTGCACCAGGACTGGCTGAACGGCAAGGAGTATAAG |
| | | TGCAAGGTGTCCAATAAGGCCCTGCCCGCCCCCATCGAGAAGA |
| | | CAATCAGCAAGGCCAAGGGCCAGCCTCGGGAGCCACAGGTGTA |
| | | CACCCTGCCTCCATCCAGAGACGAGCTGACAAAGAACCAGGTG |
| | | TCTCTGACATGTCTGGTGAAGGGCTTCTATCCTAGCGATATCGC |
| | | CGTGGAGTGGGAGTCCAATGGCCAGCCAGAGAACAATTACAAG |
| | | ACCACACCCCCTGTGCTGGACTCCGATGGCTCCTTCTTTCTGTAT |
| | | TCCAAGCTGACCGTGGATAAGTCTCGGTGGCAGCAGGGCAACG |
| | | TGTTCAGCTGTTCCGTGATGCACGAAGCCCTGCATAATCACTAT |
| | | ACTCAGAAATCCCTGTCCCTGTCACCTGGAAAGTGATAA |
| 18 | Nucleotide sequence | ATGGGCTGGTCATGCATTATTCTGTTTCTGGTCGCAACTGCTAC |
| | encoding VH2 | AGGCGTGCATAGTCAAGTGCAGCTGCAGGAGAGCGGACCTGG |
| | | CCTGGTGAAGCCATCTCAGACCCTGTCTCTCACATGTACCGTG |
| | | TCTGGATATAGCATCACCAGCGACTACGCCTGGAACTGGATCC |
| | | GGCAGCACCCCGGCAAGGGCCTGGAATGGATGGGCTACCTGT |
| | | CCTACAGCGGAGTTACATCTTATAATCCTAGCCTGAAAGGCAG |
| | | AGTGACCATCAGCAGAGATACCTCCAAGAACCAGTTCAGCCT |
| | | GAAGCTGAGCAGCGTGACAGCCGCTGACACCGCCGTGTACTA |
| | | CTGCGCCAGAAAGGGCACATACTACCGGTACGAGGGCAGCTA |
| | | CTGGTACTTCGACGTGTGGGGCCAGGGCACCACCGTCACAGT |
| | | GTCCAGCGCCAGCACCAAGGGCCCTTCCGTGTTTCCACTGGC |
| | | CCCCTCCTCTAAATCCACATCTGGCGGCACCGCCGCCCTGGG |
| | | CTGTCTGGTGAAGGACTACTTCCCAGAGCCTGTGACAGTGTC |
| | | CTGGAACTCTGGCGCCCTGACATCCGGCGTGCACACATTTCC |
| | | AGCCGTGCTGCAGAGCTCCGGCCTGTACAGCCTGTCTAGCGT |
| | | GGTGACAGTGCCCTCCTCTAGCCTGGGCACACAGACCTATAT |
| | | CTGCAACGTGAATCACAAGCCAAGCAATACCAAGGTGGACA |
| | | AGAAGGTGGAGCCCAAGTCCTGTGATAAGACACACACCTGC |
| | | CCCCCTTGTCCTGCTCCCGAGCTGCTGGGCGGCCCTAGCGTG |
| | | TTCCTGTTTCCACCCAAGCCTAAGGACACCCTGATGATCTCC |
| | | CGGACACCCGAGGTGACCTGCGTGGTGGTGGACGTGTCTCA |
| | | CGAGGATCCTGAGGTGAAGTTCAACTGGTATGTGGATGGCG |
| | | TGGAGGTGCACAATGCCAAGACCAAGCCCAGAGAGGAGCA |
| | | GTACAACTCTACATATAGGGTGGTGAGCGTGCTGACCGTGC |
| | | TGCACCAGGACTGGCTGAACGGCAAGGAGTATAAGTGCAA |
| | | GGTGTCCAATAAGGCCCTGCCCGCCCCCATCGAGAAGACA |
| | | ATCAGCAAGGCCAAGGGCCAGCCTCGGGAGCCACAGGTGT |
| | | ACACCCTGCCTCCATCCAGAGACGAGCTGACAAAGAACCA |
| | | GGTGTCTCTGACATGTCTGGTGAAGGGCTTCTATCCTAGCG |
| | | ATATCGCCGTGGAGTGGGAGTCCAATGGCCAGCCAGAGAA |
| | | CAATTACAAGACCACACCCCCTGTGCTGGACTCCGATGGC |
| | | TCCTTCTTTCTGTATTCCAAGCTGACCGTGGATAAGTCTCG |
| | | GTGGCAGCAGGGCAACGTGTTCAGCTGTTCCGTGATGCAC |
| | | GAAGCCCTGCATAATCACTATACTCAGAAATCCCTGTCCCT |
| | | GTCACCTGGAAAGTGATAA |
| 19 | Nucleotide sequence | ATGGGCTGGTCATGCATTATTCTGTTTCTGGTCGCAACTGC |
| | encoding VH3 | TACAGGCGTGCATAGTCAAGTGCAGCTGCAGGAGTCTGGA |
| | | CCTGGACTGGTGAAGCCTAGCCAGACCCTGAGCCTGACCT |
| | | GTACCGTCTCCGGCTACAGCATCACCTCCGACTACGCCTGG |
| | | AATTGGATCAGACAGCACCCCGGCAAGGGCCTCCAGTGGA |
| | | TGGGCTACCTGTCTTATTCTGGAGTGACATCTTACAACCCA |
| | | AGCCTGAAAGGCAGAATCACCATCAGCCGGGACACCAGCA |
| | | AGAACCAGTTCAGCCTGAAGCTGAGCAGCGTGACCGCCGC |
| | | TGATACAGCCGTGTACTACTGCGCCAGAAAGGGCACCTAC |
| | | TACCGGTACGAAGGCAGCTATTGGTACTTCGACGTGTGGG |
| | | GCCAGGGCACAACAGTGACCGTGTCCAGCGCCAGCACCAA |
| | | GGGCCCTTCCGTGTTTCCACTGGCCCCCTCCTCTAAATCCA |
| | | CATCTGGCGGCACCGCCGCCCTGGGCTGTCTGGTGAAGGA |
| | | CTACTTCCCAGAGCCTGTGACAGTGTCCTGGAACTCTGGCG |
| | | CCCTGACATCCGGCGTGCACACATTTCCAGCCGTGCTGCAG |
| | | AGCTCCGGCCTGTACAGCCTGTCTAGCGTGGTGACAGTGCC |
| | | CTCCTCTAGCCTGGGCACACAGACCTATATCTGCAACGTGA |
| | | ATCACAAGCCAAGCAATACCAAGGTGGACAAGAAGGTGGA |
| | | GCCCAAGTCCTGTGATAAGACACACACCTGCCCCCCTTGTC |
| | | CTGCTCCCGAGCTGCTGGGCGGCCCTAGCGTGTTCCTGTTT |
| | | CCACCCAAGCCTAAGGACACCCTGATGATCTCCCGGACAC |
| | | CCGAGGTGACCTGCGTGGTGGTGGACGTGTCTCACGAGGA |
| | | TCCTGAGGTGAAGTTCAACTGGTATGTGGATGGCGTGGAG |
| | | GTGCACAATGCCAAGACCAAGCCCAGAGAGGAGCAGTAC |
| | | AACTCTACATATAGGGTGGTGAGCGTGCTGACCGTGCTGC |
| | | ACCAGGACTGGCTGAACGGCAAGGAGTATAAGTGCAAGG |
| | | TGTCCAATAAGGCCCTGCCCGCCCCCATCGAGAAGACAAT |
| | | CAGCAAGGCCAAGGGCCAGCCTCGGGAGCCACAGGTGTA |
| | | CACCCTGCCTCCATCCAGAGACGAGCTGACAAAGAACCA |
| | | GGTGTCTCTGACATGTCTGGTGAAGGGCTTCTATCCTAGC |
| | | GATATCGCCGTGGAGTGGGAGTCCAATGGCCAGCCAGAG |
| | | AACAATTACAAGACCACACCCCCTGTGCTGGACTCCGATG |
| | | GCTCCTTCTTTCTGTATTCCAAGCTGACCGTGGATAAGTCT |
| | | CGGTGGCAGCAGGGCAACGTGTTCAGCTGTTCCGTGATGC |
| | | ACGAAGCCCTGCATAATCACTATACTCAGAAATCCCTGTC |
| | | CCTGTCACCTGGAAAGTGATAA |
| 20 | Nucleotide sequence | ATGGGCTGGTCATGCATTATTCTGTTTCTGGTCGCAACTGCT |
| | encoding VH4 | ACAGGCGTGCATAGTCAAGTGCAGCTGCAGGAGAGCGGACC |
| | | TGGCCTGGTGAAGCCTAGCCAGACCCTGTCCCTGACATGTAC |
| | | CGTGTCTGGCTACAGCATCACAAGCGACTACGCCTGGAATTG |
| | | GATCCGGCAGCACCCAGGAAAGGGCCTCCAGTGGATGGGCT |
| | | ATCTGAGCTATAGCGGCGTGACATCCTACAACCCCAGCCTGA |
| | | AAGGAAGAATCACCATCAGCAGAGATACATCTAAGAACCAG |
| | | TTTTTCCTGAAGCTGTCTAGCGTGACCGCCGCTGACACCGCC |
| | | GTGTACTACTGCGCCAGAAAGGGCACCTACTACCGGTACGAA |
| | | GGCAGCTACTGGTACTTCGACGTGTGGGGCCAGGGCACAACC |
| | | GTCACCGTGTCCAGCGCCAGCACCAAGGGCCCTTCCGTGTTTC |
| | | CACTGGCCCCCTCCTCTAAATCCACATCTGGCGGCACCGCCGC |
| | | CCTGGGCTGTCTGGTGAAGGACTACTTCCCAGAGCCTGTGACA |
| | | GTGTCCTGGAACTCTGGCGCCCTGACATCCGGCGTGCACACAT |
| | | TTCCAGCCGTGCTGCAGAGCTCCGGCCTGTACAGCCTGTCTAG |
| | | CGTGGTGACAGTGCCCTCCTCTAGCCTGGGCACACAGACCTAT |
| | | ATCTGCAACGTGAATCACAAGCCAAGCAATACCAAGGTGGAC |
| | | AAGAAGGTGGAGCCCAAGTCCTGTGATAAGACACACACCTGC |
| | | CCCCCTTGTCCTGCTCCCGAGCTGCTGGGCGGCCCTAGCGTGT |
| | | TCCTGTTTCCACCCAAGCCTAAGGACACCCTGATGATCTCCCG |
| | | GACACCCGAGGTGACCTGCGTGGTGGTGGACGTGTCTCACGA |
| | | GGATCCTGAGGTGAAGTTCAACTGGTATGTGGATGGCGTGGA |
| | | GGTGCACAATGCCAAGACCAAGCCCAGAGAGGAGCAGTACA |
| | | ACTCTACATATAGGGTGGTGAGCGTGCTGACCGTGCTGCACC |
| | | AGGACTGGCTGAACGGCAAGGAGTATAAGTGCAAGGTGTCC |
| | | AATAAGGCCCTGCCCGCCCCCATCGAGAAGACAATCAGCAAG |
| | | GCCAAGGGCCAGCCTCGGGAGCCACAGGTGTACACCCTGCCT |
| | | CCATCCAGAGACGAGCTGACAAAGAACCAGGTGTCTCTGACA |
| | | TGTCTGGTGAAGGGCTTCTATCCTAGCGATATCGCCGTGGAGT |
| | | GGGAGTCCAATGGCCAGCCAGAGAACAATTACAAGACCACAC |
| | | CCCCTGTGCTGGACTCCGATGGCTCCTTCTTTCTGTATTCCAAG |
| | | CTGACCGTGGATAAGTCTCGGTGGCAGCAGGGCAACGTGTTCA |
| | | GCTGTTCCGTGATGCACGAAGCCCTGCATAATCACTATACTCA |
| | | GAAATCCCTGTCCCTGTCACCTGGAAAGTGATAA |
| 21 | Nucleotide sequence | ATGGGCTGGTCATGTATTATTCTGTTTCTGGTCGCAACTGCTAC |
| | encoding VL1 | AGGGGTCCATAGTGATATTCAGATGACCCAGAGCCCCAGCAGC |
| | | CTCTCCGCCAGCGTGGGAGATAGAGTGACCATCACCTGTCACG |
| | | CCAACCAGAATATCGACGTCTGGCTGAGCTGGTATCAGCAGAA |
| | | ACCTGGCAAGGCCCCTAAGCTGCTGATCTACAAGACCAGCAAC |
| | | CTGCACACCGGCGTGCCATCTCGGTTCAGCGGCTCTGGCAGCG |
| | | GCACCGACTTCACATTTACAATCAGCTCTCTGCAGCCTGAGGA |
| | | CATCGCTACATACTACTGCCAGCAAGGCCAGTCCTACCCCCTG |
| | | ACCTTCGGCGGAGGCACAAAGCTGGAAATCAAGAGGACAGTG |
| | | GCCGCCCCAAGCGTGTTCATCTTTCCCCCTTCCGACGAGCAGCT |
| | | GAAGTCTGGCACCGCCAGCGTGGTGTGCCTGCTGAACAACTTCT |
| | | ACCCTCGGGAGGCCAAGGTCCAGTGGAAGGTGGATAACGCCCT |
| | | GCAGTCTGGCAATAGCCAGGAGTCCGTGACCGAGCAGGACTCT |
| | | AAGGATAGCACATATTCCCTGTCTAGCACCCTGACACTGAGCA |
| | | AGGCCGATTACGAGAAGCACAAGGTGTATGCCTGTGAAGTCAC |
| | | CCATCAGGGGCTGTCATCACCCGTCACTAAGTCATTCAATCGCG |
| | | GAGAATGCTGATAA |
| 22 | Nucleotide sequence | ATGGGCTGGTCATGTATTATTCTGTTTCTGGTCGCAACTGCTAC |
| | encoding VL2 | AGGGGTCCATAGTGATATTCAGATGACCCAGTCCCCAAGCAGC |
| | | CTCTCCGCCAGCGTGGGCGATAGAGTGACCATCACCTGTCACG |
| | | CCAACCAGAATATCGACGTGTGGCTGAGCTGGTACCAGCAAAA |
| | | GCCCGGCAAAGCCCCTAAGCTGCTGATCTACAAGACCAGCAAC |
| | | CTGCACACCGGCGTCCCCAGCCGGTTCAGCGGATCTGGCAGCG |
| | | GCACCGACTTCACCCTGACAATCAGCTCTCTGCAGCCTGAGGA |
| | | CATCGCTACATACTACTGCCAGCAGGGCCAGTCTTATCCTCTGA |
| | | CATTTGGCGGCGGAACAAAGCTGGAAATCAAGAGGACAGTGGC |
| | | CGCCCCAAGCGTGTTCATCTTTCCCCCTTCCGACGAGCAGCTGA |
| | | AGTCTGGCACCGCCAGCGTGGTGTGCCTGCTGAACAACTTCTAC |
| | | CCTCGGGAGGCCAAGGTCCAGTGGAAGGTGGATAACGCCCTGC |
| | | AGTCTGGCAATAGCCAGGAGTCCGTGACCGAGCAGGACTCTAA |
| | | GGATAGCACATATTCCCTGTCTAGCACCCTGACACTGAGCAAG |
| | | GCCGATTACGAGAAGCACAAGGTGTATGCCTGTGAAGTCACCC |
| | | ATCAGGGGCTGTCATCACCCGTCACTAAGTCATTCAATCGCGG |
| | | AGAATGCTGATAA |
| 23 | Nucleotide sequence | ATGGGCTGGTCATGTATTATTCTGTTTCTGGTCGCAACTGCTAC |
| | encoding VL3 | AGGGGTCCATAGTGATATTCAGATGACCCAGAGCCCCAGCAG |
| | | CCTGTCCGCCAGCGTCGGCGATAGAGTGACAATCACCTGTCA |
| | | CGCCAACCAGAATATCGACGTGTGGCTGTCTTGGTATCAGCA |
| | | AAAACCTGGCAACGCTCCTAAGCTCCTGATCTACAAGACCAG |
| | | CAACCTGCACACAGGCGTGCCAAGCCGGTTCAGCGGCAGCGG |
| | | ATCTGGCACCGACTTCACCTTCACAATCAGCTCTCTGCAGCCTG |
| | | AGGACATCGCCACCTACTACTGCCAGCAGGGCCAGTCCTACCC |
| | | CCTGACCTTTGGCGGAGGCACAAAGCTGGAAATCAAGAGGACA |
| | | GTGGCCGCCCCAAGCGTGTTCATCTTTCCCCCTTCCGACGAGCA |
| | | GCTGAAGTCTGGCACCGCCAGCGTGGTGTGCCTGCTGAACAACT |
| | | TCTACCCTCGGGAGGCCAAGGTCCAGTGGAAGGTGGATAACGC |
| | | CCTGCAGTCTGGCAATAGCCAGGAGTCCGTGACCGAGCAGGAC |
| | | TCTAAGGATAGCACATATTCCCTGTCTAGCACCCTGACACTGAG |
| | | CAAGGCCGATTACGAGAAGCACAAGGTGTATGCCTGTGAAGTC |
| | | ACCCATCAGGGGCTGTCATCACCCGTCACTAAGTCATTCAATCG |
| | | CGGAGAATGCTGATAA |
| 24 | Nucleotide sequence | ATGGGCTGGTCATGTATTATTCTGTTTCTGGTCGCAACTGCTA |
| | encoding VL4 | CAGGGGTCCATAGTGATATTCAGATGACCCAGTCTCCATCTA |
| | | GCCTCTCCGCCAGCGTGGGCGACCGGGTGACCATCACCTGTCA |
| | | CGCCAACCAGAACATCGACGTGTGGCTGAGCTGGTATCAGCA |
| | | GAAACCTGGAAATGCCCCTAAGCTGCTGATCTACAAGACCAG |
| | | CAACCTGCACACCGGCGTCCCCAGCAGATTCAGCGGCAGCGG |
| | | CTCTGGCACCGACTTCACCCTGACAATCAGCAGCCTGCAGCCT |
| | | GAGGATATCGCTACATACTACTGCCAGCAAGGCCAGTCCTACC |
| | | CCCTGACATTTGGCGGCGGAACAAAGCTGGAAATCAAGAGGA |
| | | CAGTGGCCGCCCCAAGCGTGTTCATCTTTCCCCCTTCCGACGA |
| | | GCAGCTGAAGTCTGGCACCGCCAGCGTGGTGTGCCTGCTGAA |
| | | CAACTTCTACCCTCGGGAGGCCAAGGTCCAGTGGAAGGTGGA |
| | | TAACGCCCTGCAGTCTGGCAATAGCCAGGAGTCCGTGACCGA |
| | | GCAGGACTCTAAGGATAGCACATATTCCCTGTCTAGCACCCT |
| | | GACACTGAGCAAGGCCGATTACGAGAAGCACAAGGTGTATG |
| | | CCTGTGAAGTCACCCATCAGGGGCTGTCATCACCCGTCACTA |
| | | AGTCATTCAATCGCGGAGAATGCTGATAA |
| 25 | VH | QVQLQESGPGLVKPSQSLSLTCTVTGYSITSDYAWNWIRQFPGN |
| | | KLQWMGYLSYSGVTSYNPSLKGRISITRDTSKNQFFLQLSSVTP |
| | | EDTATYYCARKGTYYRYEGSYWYFDVWGAGTTVTVSS |
| 26 | VL | DIQMNQSPSSLSASLGDTITITCHANQNIDVWLSWYQQKPGNV |
| | | PKLLIYKTSNLHTGVPSRFSGSGSGTDFTLTISSLQPEDIATYYC |
| | | QQGQSYPLTFGGGTKLEIK |
| 27 | Nucleotide sequence | ATGGGCTGGTCATGCATTATTCTGTTTCTGGTCGCAACTGC |
| | encoding VH | TACAGGCGTGCATAGTCAGGTGCAGCTGCAGGAGAGCGGA |
| | | CCTGGCCTGGTGAAGCCTAGCCAGAGCCTGTCCCTCACCTG |
| | | TACCGTGACCGGCTACTCCATCACAAGCGACTACGCCTGGA |
| | | ATTGGATCAGACAGTTTCCAGGCAACAAGCTGCAGTGGATG |
| | | GGCTACCTGTCTTATTCTGGCGTGACATCTTACAACCCCAGC |
| | | CTGAAAGGCAGAATCAGCATCACCCGGGACACCAGCAAGAA |
| | | CCAGTTCTTCCTGCAACTGAGCAGCGTGACCCCTGAAGATA |
| | | CCGCCACATACTACTGCGCCAGAAAGGGCACCTACTACCGG |
| | | TACGAGGGCAGCTATTGGTACTTCGACGTGTGGGGCGCTGG |
| | | AACAACCGTCACAGTGTCCAGCGCCAGCACCAAGGGCCCTT |
| | | CCGTGTTTCCACTGGCCCCCTCCTCTAAATCCACATCTGGCG |
| | | GCACCGCCGCCCTGGGCTGTCTGGTGAAGGACTACTTCCCA |
| | | GAGCCTGTGACAGTGTCCTGGAACTCTGGCGCCCTGACATCC |
| | | GGCGTGCACACATTTCCAGCCGTGCTGCAGAGCTCCGGCCT |
| | | GTACAGCCTGTCTAGCGTGGTGACAGTGCCCTCCTCTAGCC |
| | | TGGGCACACAGACCTATATCTGCAACGTGAATCACAAGCC |
| | | AAGCAATACCAAGGTGGACAAGAAGGTGGAGCCCAAGTC |
| | | CTGTGATAAGACACACACCTGCCCCCCTTGTCCTGCTCCCG |
| | | AGCTGCTGGGCGGCCCTAGCGTGTTCCTGTTTCCACCCAAG |
| | | CCTAAGGACACCCTGATGATCTCCCGGACACCCGAGGTGA |
| | | CCTGCGTGGTGGTGGACGTGTCTCACGAGGATCCTGAGGT |
| | | GAAGTTCAACTGGTATGTGGATGGCGTGGAGGTGCACAAT |
| | | GCCAAGACCAAGCCCAGAGAGGAGCAGTACAACTCTACA |
| | | TATAGGGTGGTGAGCGTGCTGACCGTGCTGCACCAGGACT |
| | | GGCTGAACGGCAAGGAGTATAAGTGCAAGGTGTCCAATA |
| | | AGGCCCTGCCCGCCCCCATCGAGAAGACAATCAGCAAGG |
| | | CCAAGGGCCAGCCTCGGGAGCCACAGGTGTACACCCTGC |
| | | CTCCATCCAGAGACGAGCTGACAAAGAACCAGGTGTCTCT |
| | | GACATGTCTGGTGAAGGGCTTCTATCCTAGCGATATCGCC |
| | | GTGGAGTGGGAGTCCAATGGCCAGCCAGAGAACAATTAC |
| | | AAGACCACACCCCCTGTGCTGGACTCCGATGGCTCCTTCT |
| | | TTCTGTATTCCAAGCTGACCGTGGATAAGTCTCGGTGGCA |
| | | GCAGGGCAACGTGTTCAGCTGTTCCGTGATGCACGAAGC |
| | | CCTGCATAATCACTATACTCAGAAATCCCTGTCCCTGTCA |
| | | CCTGGAAAGTGATAA |
| 28 | Nucleotide sequence | ATGGGCTGGTCATGTATTATTCTGTTTCTGGTCGCAACTG |
| | encoding VL | CTACAGGGGTCCATAGTGATATCCAGATGAACCAGAGCC |
| | | CTTCTAGCCTCTCCGCCAGCCTGGGCGACACCATTACAAT |
| | | CACCTGTCACGCTAATCAGAACATCGACGTGTGGCTGAG |
| | | CTGGTATCAGCAGAAACCTGGCAACGTGCCCAAGCTGCT |
| | | GATCTACAAGACCAGCAACCTGCACACCGGCGTGCCATC |
| | | TAGATTCAGCGGATCTGGCAGCGGCACCGACTTCACCCTG |
| | | ACCATCAGCAGCCTGCAGCCTGAGGATATCGCCACATAC |
| | | TACTGCCAGCAAGGCCAGTCCTACCCCCTGACATTTGGCG |
| | | GCGGAACAAAGCTGGAAATCAAGAGGACAGTGGCCGCCC |
| | | CAAGCGTGTTCATCTTTCCCCCTTCCGACGAGCAGCTGAAG |
| | | TCTGGCACCGCCAGCGTGGTGTGCCTGCTGAACAACTTCT |
| | | ACCCTCGGGAGGCCAAGGTCCAGTGGAAGGTGGATAACG |
| | | CCCTGCAGTCTGGCAATAGCCAGGAGTCCGTGACCGAGC |
| | | AGGACTCTAAGGATAGCACATATTCCCTGTCTAGCACCC |
| | | TGACACTGAGCAAGGCCGATTACGAGAAGCACAAGGTG |
| | | TATGCCTGTGAAGTCACCCATCAGGGGCTGTCATCACCC |
| | | GTCACTAAGTCATTCAATCGCGGAGAATGCTGATAA |
| 29 | VH FR1 | QVQLQESGPGLVKPSQSLSLTCTVT |
| 30 | VH FR2 | WIRQFPGNKLQWMG |
| 31 | VH FR3 | RISITRDTSKNQFFLQLSSVTPEDTATYYCAR |
| 32 | VH FR4 | WGAGTTVTVSS |
| 33 | VH1/VH2/VH3/VH4 | QVQLQESGPGLVKPSQTLSLTCTVS |
| | FR1 | |
| 34 | VH1 FR2 | WIRQHPGKGLEWIG |
| 35 | VH1 FR3 | RVTISVDTSKNQFSLKLSSVTAADTAVYYCAR |
| 36 | VH1/VH2/VH3/VH4 | WGQGTTVTVSS |
| | FR4 | |
| 37 | VH2 FR2 | WIRQHPGKGLEWMG |
| 38 | VH2 FR3 | RVTISRDTSKNQFSLKLSSVTAADTAVYYCAR |
| 39 | VH3/VH4 FR2 | WIRQHPGKGLQWMG |
| 40 | VH3 FR3 | RITISRDTSKNQFSLKLSSVTAADTAVYYCAR |
| 41 | VH4 FR3 | RITISRDTSKNQFFLKLSSVTAADTAVYYCAR |
| 42 | VL FR1 | DIQMNQSPSSLSASLGDTITITC |
| 43 | VL FR2 | WYQQKPGNVPKLLIY |
| 44 | VL/VL2/VL4 FR3 | GVPSRFSGSGSGTDFTLTISSLQPEDIATYYC |
| 45 | Light chain FR4 | FGGGTKLEIK |
| 46 | VL1/VL2/VL3/VL4 | DIQMTQSPSSLSASVGDRVTITC |
| | FR1 | |
| 47 | VL1/VL2 FR2 | WYQQKPGKAPKLLIY |
| 48 | VL1/VL3 FR3 | GVPSRFSGSGSGTDFTFTISSLQPEDIATYYC |
| 49 | VL3/VL4 FR2 | WYQQKPGNAPKLLIY |
| 50 | VH1 | MGWSCIILFLVATATGVHSQVQLQESGPGLVKPSQTLSLT |
| | | CTVSGYSITSDYAWNWIRQHPGKGLEWIGYLSYSGVTSY |
| | | NPSLKGRVTISVDTSKNQFSLKLSSVTAADTAVYYCARK |
| | | GTYYRYEGSYWYFDVWGQGTTVTVSSASTKGPSVFPLA |
| | | PSSKSTSGGTAALGCLVKDYFPEPVTVSWNSGALTSGVH |
| | | TFPAVLQSSGLYSLSSVVTVPSSSLGTQTYICNVNHKPSN |
| | | TKVDKKVEPKSCDKTHTCPPCPAPELLGGPSVFLFPPKPK |
| | | DTLMISRTPEVTCVVVDVSHEDPEVKFNWYVDGVEVHN |
| | | AKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFF |
| | | LYSKLTVDKSRWQQGNVFSCSVMHEALHNHYTQKSLSL |
| | | SPGK |
| 51 | VH2 | MGWSCIILFLVATATGVHSQVQLQESGPGLVKPSQTLSLT |
| | | CTVSGYSITSDYAWNWIRQHPGKGLEWMGYLSYSGVTS |
| | | YNPSLKGRVTISRDTSKNQFSLKLSSVTAADTAVYYCAR |
| | | KGTYYRYEGSYWYFDVWGQGTTVTVSSASTKGPSVFPL |
| | | APSSKSTSGGTAALGCLVKDYFPEPVTVSWNSGALTSGV |
| | | HTFPAVLQSSGLYSLSSVVTVPSSSLGTQTYICNVNHKPS |
| | | NTKVDKKVEPKSCDKTHTCPPCPAPELLGGPSVFLFPPKP |
| | | KDTLMISRTPEVTCVVVDVSHEDPEVKFNWYVDGVEVH |
| | | NAKTKPREEQYNSTYRVVSVLTVLHQDWLNGKEYKCK |
| | | VSNKALPAPIEKTISKAKGQPREPQVYTLPPSRDELTKNQ |
| | | VSLTCLVKGFYPSDIAVEWESNGQPENNYKTTPPVLDSDG |
| | | SFFLYSKLTVDKSRWQQGNVFSCSVMHEALHNHYTQKS |
| | | LSLSPGK |
| 52 | VH3 | MGWSCIILFLVATATGVHSQVQLQESGPGLVKPSQTLSLT |
| | | CTVSGYSITSDYAWNWIRQHPGKGLQWMGYLSYSGVTS |
| | | YNPSLKGRITISRDTSKNQFSLKLSSVTAADTAVYYCARK |
| | | GTYYRYEGSYWYFDVWGQGTTVTVSSASTKGPSVFPLA |
| | | PSSKSTSGGTAALGCLVKDYFPEPVTVSWNSGALTSGVH |
| | | TFPAVLQSSGLYSLSSVVTVPSSSLGTQTYICNVNHKPSN |
| | | TKVDKKVEPKSCDKTHTCPPCPAPELLGGPSVFLFPPKPK |
| | | DTLMISRTPEVTCVVVDVSHEDPEVKFNWYVDGVEVHN |
| | | AKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFF |
| | | LYSKLTVDKSRWQQGNVFSCSVMHEALHNHYTQKSLSL |
| | | SPGK |
| 53 | VH4 | MGWSCIILFLVATATGVHSQVQLQESGPGLVKPSQTLSLT |
| | | CTVSGYSITSDYAWNWIRQHPGKGLQWMGYLSYSGVTS |
| | | YNPSLKGRITISRDTSKNQFFLKLSSVTAADTAVYYCARK |
| | | GTYYRYEGSYWYFDVWGQGTTVTVSSASTKGPSVFPLAP |
| | | SSKSTSGGTAALGCLVKDYFPEPVTVSWNSGALTSGVHT |
| | | FPAVLQSSGLYSLSSVVTVPSSSLGTQTYICNVNHKPSNTK |
| | | VDKKVEPKSCDKTHTCPPCPAPELLGGPSVFLFPPKPKDTL |
| | | MISRTPEVTCVVVDVSHEDPEVKFNWYVDGVEVHNAKTK |
| | | PREEQYNSTYRVVSVLTVLHQDWLNGKEYKCKVSNKALP |
| | | APIEKTISKAKGQPREPQVYTLPPSRDELTKNQVSLTCLVK |
| | | GFYPSDIAVEWESNGQPENNYKTTPPVLDSDGSFFLYSKLT |
| | | VDKSRWQQGNVFSCSVMHEALHNHYTQKSLSLSPGK |
| 54 | VH5 | MGWSCIILFLVATATGVHSQVQLQESGPGLVKPSQTLSLT |
| | | CTVSGYSITSDYAWNWIRQHPGKGLEWMGYLSYSGVTS |
| | | YNPSLKGRVTISRDTSKNQFSLKLSSVTAADTAVYYCARK |
| | | GTYYRYEGSYWYFDVWGQGTTVTVSSASTKGPSVFPLAP |
| | | SSKSTSGGTAALGCLVKDYFPEPVTVSWNSGALTSGVHTF |
| | | PAVLQSSGLYSLSSVVTVPSSSLGTQTYICNVNHKPSNTKV |
| | | DKKVEPKSCDKTHTCPPCPAPEAAGGPSVFLFPPKPKDTL |
| | | MISRTPEVTCVVVDVSHEDPEVKFNWYVDGVEVHNAKT |
| | | KPREEQYNSTYRVVSVLTVLHQDWLNGKEYKCKVSNK |
| | | ALPAPIEKTISKAKGQPREPQVYTLPPSRDELTKNQVSLTC |
| | | LVKGFYPSDIAVEWESNGQPENNYKTTPPVLDSDGSFFLY |
| | | SKLTVDKSRWQQGNVFSCSVMHEALHNHYTQKSLSLSPGK |
| 55 | VH6 | MGWSCIILFLVATATGVHSQVQLQESGPGLVKPSQTLSLT |
| | | CTVSGYSITSDYAWNWIRQHPGKGLEWMGYLSYSGVTSY |
| | | NPSLKGRVTISRDTSKNQFSLKLSSVTAADTAVYYCARKG |
| | | TYYRYEGSYWYFDVWGQGTTVTVSSASTKGPSVFPLAPS |
| | | SKSTSGGTAALGCLVKDYFPEPVTVSWNSGALTSGVHTFP |
| | | AVLQSSGLYSLSSVVTVPSSSLGTQTYICNVNHKPSNTKVD |
| | | KKVEPKSCDKTHTCPPCPAPELLGGPSVFLFPPKPKDTLMI |
| | | SRTPEVTCVVVDVSHEDPEVKFNWYVDGVEVHNAKTKPR |
| | | EEQYASTYRVVSVLTVLHQDWLNGKEYKCKVSNKALPAPI |
| | | EKTISKAKGQPREPQVYTLPPSRDELTKNQVSLTCLVKGFY |
| | | PSDIAVEWESNGQPENNYKTTPPVLDSDGSFFLYSKLTVDK |
| | | SRWQQGNVFSCSVMHEALHNHYTQKSLSLSPGK |
| 56 | VH7 | MGWSCIILFLVATATGVHSQVQLQESGPGLVKPSQTLSLT |
| | | CTVSGYSITSDYAWNWIRQHPGKGLEWMGYLSYSGVTS |
| | | YNPSLKGRVTISRDTSKNQFSLKLSSVTAADTAVYYCARK |
| | | GTYYRYEGSYWYFDVWGQGTTVTVSSASTKGPSVFPLAP |
| | | CSRSTSESTAALGCLVKDYFPEPVTVSWNSGALTSGVHTF |
| | | PAVLQSSGLYSLSSVVTVPSSSLGTKTYTCNVDHKPSNTK |
| | | VDKRVESKYGPPCPPCPAPEFLGGPSVFLFPPKPKDTLMIS |
| | | RTPEVTCVVVDVSQEDPEVQFNWYVDGVEVHNAKTKPR |
| | | EEQFNSTYRVVSVLTVLHQDWLNGKEYKCKVSNKGLPSS |
| | | IEKTISKAKGQPREPQVYTLPPSQEEMTKNQVSLTCLVKG |
| | | FYPSDIAVEWESNGQPENNYKTTPPVLDSDGSFFLYSRLT |
| | | VDKSRWQEGNVFSCSVMHEALHNHYTQKSLSLSLGK |
| 57 | VL1 | MGWSCIILFLVATATGVHSDIQMTQSPSSLSASVGDRVTI |
| | | TCHANQNIDVWLSWYQQKPGKAPKLLIYKTSNLHTGVP |
| | | SRFSGSGSGTDFTFTISSLQPEDIATYYCQQGQSYPLTFGG |
| | | GTKLEIKRTVAAPSVFIFPPSDEQLKSGTASVVCLLNNFYP |
| | | REAKVQWKVDNALQSGNSQESVTEQDSKDSTYSLSSTLT |
| | | LSKADYEKHKVYACEVTHQGLSSPVTKSFNRGEC |
| 58 | VL2 | MGWSCIILFLVATATGVHSDIQMTQSPSSLSASVGDRVTI |
| | | TCHANQNIDVWLSWYQQKPGKAPKLLIYKTSNLHTGVP |
| | | SRFSGSGSGTDFTLTISSLQPEDIATYYCQQGQSYPLTFGG |
| | | GTKLEIKRTVAAPSVFIFPPSDEQLKSGTASVVCLLNNFYP |
| | | REAKVQWKVDNALQSGNSQESVTEQDSKDSTYSLSSTLT |
| | | LSKADYEKHKVYACEVTHQGLSSPVTKSFNRGEC |
| 59 | VL3 | MGWSCIILFLVATATGVHSDIQMTQSPSSLSASVGDRVTI |
| | | TCHANQNIDVWLSWYQQKPGNAPKLLIYKTSNLHTGVP |
| | | SRFSGSGSGTDFTFTISSLQPEDIATYYCQQGQSYPLTFGG |
| | | GTKLEIKRTVAAPSVFIFPPSDEQLKSGTASVVCLLNNFY |
| | | PREAKVQWKVDNALQSGNSQESVTEQDSKDSTYSLSST |
| | | LTLSKADYEKHKVYACEVTHQGLSSPVTKSFNRGEC |
| 60 | VL4 | MGWSCIILFLVATATGVHSDIQMTQSPSSLSASVGDRVTI |
| | | TCHANQNIDVWLSWYQQKPGNAPKLLIYKTSNLHTGVP |
| | | SRFSGSGSGTDFTLTISSLQPEDIATYYCQQGQSYPLTFG |
| | | GGTKLEIKRTVAAPSVFIFPPSDEQLKSGTASVVCLLNNF |
| | | YPREAKVQWKVDNALQSGNSQESVTEQDSKDSTYSLSS |
| | | TLTLSKADYEKHKVYACEVTHQGLSSPVTKSFNRGEC |
| 61 | Nucleotide sequence | ATGGGCTGGTCATGCATTATTCTGTTTCTGGTCGCAAC |
| | encoding VH5 | TGCTACAGGCGTGCATAGTCAAGTGCAGCTGCAGGAG |
| | | AGCGGACCTGGCCTGGTGAAGCCATCTCAGACCCTGT |
| | | CTCTCACATGTACCGTGTCTGGATATAGCATCACCAGC |
| | | GACTACGCCTGGAACTGGATCCGGCAGCACCCCGGCA |
| | | AGGGCCTGGAATGGATGGGCTACCTGTCCTACAGCGG |
| | | AGTTACATCTTATAATCCTAGCCTGAAAGGCAGAGTGA |
| | | CCATCAGCAGAGATACCTCCAAGAACCAGTTCAGCCT |
| | | GAAGCTGAGCAGCGTGACAGCCGCTGACACCGCCGTG |
| | | TACTACTGCGCCAGAAAGGGCACATACTACCGGTACGA |
| | | GGGCAGCTACTGGTACTTCGACGTGTGGGGCCAGGGCA |
| | | CCACCGTCACAGTGTCCAGCGCCAGCACAAAGGGCCC |
| | | TAGCGTGTTTCCACTGGCCCCCTCCTCTAAGTCCACCT |
| | | CTGGAGGAACAGCCGCCCTGGGCTGTCTGGTGAAGGAT |
| | | TATTTCCCAGAGCCCGTGACCGTGTCCTGGAACTCTGGC |
| | | GCCCTGACCAGCGGAGTGCACACATTTCCAGCCGTGCTG |
| | | CAGAGCTCCGGACTGTACTCCCTGTCTAGCGTGGTGACC |
| | | GTGCCTTCCTCTAGCCTGGGCACCCAGACATATATCTGC |
| | | AACGTGAATCACAAGCCCTCCAATACAAAGGTGGACAA |
| | | GAAGGTGGAGCCTAAGTCTTGTGATAAGACCCACACATG |
| | | CCCCCCTTGTCCTGCACCAGAGGCAGCAGGAGGACCTT |
| | | CCGTGTTCCTGTTTCCACCCAAGCCAAAGGACACCCTG |
| | | ATGATCAGCCGCACCCCTGAGGTGACATGCGTGGTGGT |
| | | GGACGTGTCCCACGAGGATCCAGAGGTGAAGTTTAACT |
| | | GGTACGTGGATGGCGTGGAGGTGCACAATGCCAAGACC |
| | | AAGCCTCGGGAGGAGCAGTACAACTCTACCTATAGAGTG |
| | | GTGAGCGTGCTGACAGTGCTGCACCAGGACTGGCTGAAC |
| | | GGCAAGGAGTATAAGTGCAAGGTGTCTAATAAGGCCCT |
| | | GCCCGCCCCTATCGAGAAGACCATCAGCAAGGCAAAGG |
| | | GACAGCCTAGGGAGCCACAGGTGTACACACTGCCTCCA |
| | | TCTAGAGACGAGCTGACCAAGAACCAGGTGAGCCTGACA |
| | | TGTCTGGTGAAGGGCTTCTATCCAAGCGATATCGCCGTG |
| | | GAGTGGGAGTCCAATGGCCAGCCCGAGAACAATTACAAG |
| | | ACCACACCCCCTGTGCTGGACAGCGATGGCTCCTTCTTTC |
| | | TGTATTCCAAGCTGACCGTGGATAAGTCTCGGTGGCAGC |
| | | AGGGCAACGTGTTTAGCTGTTCCGTGATGCACGAGGCCC |
| | | TGCACAATCACTACACCCAGAAGTCTCTGAGCCTGTCC |
| | | CCCGGCAAGTGA |
| 62 | Nucleotide sequence | ATGGGCTGGTCATGCATTATTCTGTTTCTGGTCGCAAC |
| | encoding VH6 | TGCTACAGGCGTGCATAGTCAAGTGCAGCTGCAGGAGA |
| | | GCGGACCTGGCCTGGTGAAGCCATCTCAGACCCTGTCT |
| | | CTCACATGTACCGTGTCTGGATATAGCATCACCAGCGAC |
| | | TACGCCTGGAACTGGATCCGGCAGCACCCCGGCAAGGG |
| | | CCTGGAATGGATGGGCTACCTGTCCTACAGCGGAGTTA |
| | | CATCTTATAATCCTAGCCTGAAAGGCAGAGTGACCATC |
| | | AGCAGAGATACCTCCAAGAACCAGTTCAGCCTGAAGCT |
| | | GAGCAGCGTGACAGCCGCTGACACCGCCGTGTACTACT |
| | | GCGCCAGAAAGGGCACATACTACCGGTACGAGGGCAG |
| | | CTACTGGTACTTCGACGTGTGGGGCCAGGGCACCACCG |
| | | TCACAGTGTCCAGCGCCAGCACCAAGGGCCCTTCCGTG |
| | | TTTCCACTGGCCCCCTCCTCTAAATCCACATCTGGCGGC |
| | | ACCGCCGCCCTGGGCTGTCTGGTGAAGGACTACTTCCC |
| | | AGAGCCTGTGACAGTGTCCTGGAACTCTGGCGCCCTGA |
| | | CATCCGGCGTGCACACATTTCCAGCCGTGCTGCAGAGCT |
| | | CCGGCCTGTACAGCCTGTCTAGCGTGGTGACAGTGCCC |
| | | TCCTCTAGCCTGGGCACACAGACCTATATCTGCAACGT |
| | | GAATCACAAGCCAAGCAATACCAAGGTGGACAAGAAG |
| | | GTGGAGCCCAAGTCCTGTGATAAGACACACACCTGCCC |
| | | CCCTTGTCCTGCTCCCGAGCTGCTGGGCGGCCCTAGCG |
| | | TGTTCCTGTTTCCACCCAAGCCTAAGGACACCCTGATG |
| | | ATCTCCCGGACACCCGAGGTGACCTGCGTGGTGGTGGA |
| | | CGTGTCTCACGAGGATCCTGAGGTGAAGTTCAACTGGT |
| | | ATGTGGATGGCGTGGAGGTGCACAATGCCAAGACCAA |
| | | GCCCAGAGAGGAGCAGTACGCCTCTACATATAGGGTG |
| | | GTGAGCGTGCTGACCGTGCTGCACCAGGACTGGCTGAA |
| | | CGGCAAGGAGTATAAGTGCAAGGTGTCCAATAAGGCC |
| | | CTGCCCGCCCCCATCGAGAAGACAATCAGCAAGGCCA |
| | | AGGGCCAGCCTCGGGAGCCACAGGTGTACACCCTGCCT |
| | | CCATCCAGAGACGAGCTGACAAAGAACCAGGTGTCTC |
| | | TGACATGTCTGGTGAAGGGCTTCTATCCTAGCGATATC |
| | | GCCGTGGAGTGGGAGTCCAATGGCCAGCCAGAGAACA |
| | | ATTACAAGACCACACCCCCTGTGCTGGACTCCGATGGC |
| | | TCCTTCTTTCTGTATTCCAAGCTGACCGTGGATAAGTCT |
| | | CGGTGGCAGCAGGGCAACGTGTTCAGCTGTTCCGTGAT |
| | | GCACGAAGCCCTGCATAATCACTATACTCAGAAATCCC |
| | | TGTCCCTGTCACCTGGAAAGTGA |
| 63 | Nucleotide sequence | ATGGGCTGGTCTTGTATTATTCTGTTTCTGGTCGCAAC |
| | encoding VH7 | TGCTACAGGCGTGCATTCTCAAGTGCAGCTGCAGGAG |
| | | AGCGGACCTGGCCTGGTGAAGCCATCTCAGACCCTGT |
| | | CTCTCACATGTACCGTGTCTGGATATAGCATCACCAGC |
| | | GACTACGCCTGGAACTGGATCCGGCAGCACCCCGGCA |
| | | AGGGCCTGGAATGGATGGGCTACCTGTCCTACAGCGG |
| | | AGTTACATCTTATAATCCTAGCCTGAAAGGCAGAGTG |
| | | ACCATCAGCAGAGATACCTCCAAGAACCAGTTCAGCC |
| | | TGAAGCTGAGCAGCGTGACAGCCGCTGACACCGCCGT |
| | | GTACTACTGCGCCAGAAAGGGCACATACTACCGGTAC |
| | | GAGGGCAGCTACTGGTACTTCGACGTGTGGGGCCAGG |
| | | GCACCACCGTCACAGTGTCCAGCGCCAGCACCAAGGG |
| | | CCCTTCCGTGTTTCCCCTGGCCCCTTGCTCCCGGTCCAC |
| | | ATCTGAGAGCACCGCCGCCCTGGGCTGTCTGGTGAAG |
| | | GACTACTTCCCAGAGCCCGTGACCGTGAGCTGGAACA |
| | | GCGGCGCCCTGACAAGCGGCGTGCACACATTTCCCGC |
| | | CGTGCTGCAGAGCTCCGGCCTGTACTCCCTGTCTAGCG |
| | | TGGTGACAGTGCCTTCCTCTAGCCTGGGCACCAAGACA |
| | | TATACCTGTAACGTGGACCACAAGCCAAGCAATACCA |
| | | AGGTGGATAAGCGGGTGGAGTCTAAGTACGGCCCTCC |
| | | TTGCCCTCCATGTCCTGCTCCAGAGTTTCTGGGCGGCC |
| | | CTTCCGTGTTCCTGTTTCCACCCAAACCAAAGGACACA |
| | | CTGATGATCTCTAGAACACCAGAGGTGACCTGCGTGGT |
| | | GGTGGACGTGAGCCAGGAGGATCCCGAGGTGCAGTTC |
| | | AACTGGTACGTGGATGGCGTGGAGGTGCACAATGCCA |
| | | AGACCAAGCCAAGAGAGGAGCAGTTTAACTCTACATA |
| | | CAGGGTGGTGAGCGTGCTGACCGTGCTGCACCAGGAT |
| | | TGGCTCAACGGCAAGGAGTATAAGTGCAAGGTGTCCA |
| | | ATAAGGGCCTGCCCTCCTCTATCGAGAAGACAATCTCT |
| | | AAGGCTAAGGGCCAGCCAAGAGAGCCTCAGGTGTACA |
| | | CCCTGCCTCCAAGCCAGGAGGAGATGACAAAGAACCA |
| | | GGTGTCCCTGACATGTCTGGTGAAGGGCTTCTATCCCT |
| | | CCGACATCGCCGTGGAGTGGGAGTCTAATGGCCAGCC |
| | | TGAGAACAATTACAAGACCACACCCCCTGTGCTGGAC |
| | | TCTGATGGCAGCTTCTTTCTGTATTCCAGGCTGACCGT |
| | | GGATAAGTCTCGGTGGCAGGAGGGCAACGTGTTCAGC |
| | | TGCTCTGTGATGCACGAAGCCCTGCATAATCACTATA |
| | | CTCAGAAAAGTCTGTCACTGTCACTGGGAAAGTGA |

What is claimed is:

1. A method of treating stroke, comprising administering to a subject an effective amount of a humanized monoclonal antibody or antigen-binding fragment thereof specific to a transient receptor potential melastatin 4 (TRPM4) protein, wherein the antibody or antigen-binding fragment thereof comprises a heavy chain variable region and a light chain variable region, wherein the heavy chain variable region comprises a CDR1-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 1, a CDR2-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 2 and a CDR3-H domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 3, and wherein the light chain variable region comprises a CDR1-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 4, a CDR2-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 5 and a CDR3-L domain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 6.

2. The method of claim 1, wherein the heavy chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 7, SEQ ID NO: 8, SEQ ID NO: 9, or SEQ ID NO: 10, and wherein the light chain variable region comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 11, SEQ ID NO: 12, SEQ ID NO: 13, or SEQ ID NO: 14.

3. The method of claim 1, wherein the antibody or antigen-binding fragment thereof comprises:
a) a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11;
b) a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12;
c) a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 13;
d) a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 7, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 14;
e) a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 11; or
f) a heavy chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 8, and a light chain variable region comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 12.

4. The method of claim 1, wherein the antibody or antigen-binding fragment thereof comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, SEQ ID NO: 51, SEQ ID NO: 52, SEQ ID NO: 53, SEQ ID NO: 54, SEQ ID NO: 55, or SEQ ID NO: 56.

5. The method of claim 1, wherein the antibody or antigen-binding fragment thereof comprises a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57, SEQ ID NO: 58, SEQ ID NO: 59, or SEQ ID NO: 60.

6. The method of claim 1, wherein the antibody or antigen-binding fragment thereof comprises a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, SEQ ID NO: 51, SEQ ID NO: 52, SEQ ID NO: 53, SEQ ID NO: 54, SEQ ID NO: 55, or SEQ ID NO: 56, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57, SEQ ID NO: 58, SEQ ID NO: 59, or SEQ ID NO: 60.

7. The method of claim 1, wherein the antibody or antigen-binding fragment thereof comprises:
a) a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57;
b) a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having 98%, or 99% sequence identity to SEQ ID NO: 58;
c) a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 59;
d) a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 50, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 60;
e) a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57;
f) a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 51, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 58;
g) a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 54, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57;
h) a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 55, and a light chain comprising an amino acid sequence having 98%, or 99% sequence identity to SEQ ID NO: 57; or
i) a heavy chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 56, and a light chain comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 57.

8. The method of claim 1, wherein the antibody specifically binds to a peptide comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 15; or a peptide comprising an amino acid sequence having at least 85%, 86%, 87%, 88%, 89%, 90%, 91%, 92%, 93%, 94%, 95%, 96%, 97%, 98%, or 99% sequence identity to SEQ ID NO: 16.

9. The method of claim 1, wherein the antibody inhibits TRPM4 activity.

10. The method of claim 1, wherein the antibody inhibits TRPM4 current and/or internalizes membrane TRPM4 protein.

11. The method of claim 1, wherein the antibody or antigen-binding fragment thereof is encoded by a nucleic acid comprising a polynucleotide sequence having at least 85%, 86%, 87%, 88%, 89%, 90%, 91%, 92%, 93%, 94%, 95%, 96%, 97%, 98%, or 99% sequence identity to any one of SEQ ID NOs: 17-24 and SEQ ID NOs: 61-63.

12. The method of claim 1, wherein the stroke is selected from the group consisting of hemorrhagic stroke and ischemic stroke, wherein optionally the stroke is ischemic stroke.

13. The method of claim 1, wherein the effective amount of the antibody or antigen-binding fragment thereof is from 0.1 mg/kg to 15 mg/kg, or from 0.2 mg/kg to 14 mg/kg, or from 0.4 mg/kg to 13 mg/kg, or from 0.6 mg/kg to 12 mg/kg, or from 0.8 mg/kg to 11 mg/kg, or from 1 mg/kg to 10 mg/kg, or from 2 mg/kg to 9 mg/kg, or from 3 mg/kg to 8 mg/kg, or from 4 mg/kg to 7 mg/kg, or about 0.1 mg/kg, or about 0.2 mg/kg, or about 0.4 mg/kg, or about 0.6 mg/kg, or about 0.8 mg/kg, or about 1 mg/kg, or about 2 mg/kg, or about 3 mg/kg, or about 4 mg/kg, or about 5 mg/kg, or about 6 mg/kg, or about 7 mg/kg, or about 8 mg/kg, or about 9 mg/kg, or about 10 mg/kg, or about 11 mg/kg, or about 12 mg/kg, or about 13 mg/kg, or about 14 mg/kg, or about 15 mg/kg, wherein optionally the effective amount of the antibody or antigen-binding fragment thereof is about 1 mg/kg.

14. The method of claim 13, wherein the effective amount of the antibody or antigen-binding fragment thereof is administered within about 0.5 hour, about 1.0 hour, about 1.5 hours, about 2.0 hours, about 2.5 hours, about 3.0 hours, about 3.5 hours, about 4.0 hours, about 4.5 hours, about 5.0 hours, about 5.5 hours, about 6.0 hours, or about 6.5 hours after the onset of the first stroke symptom(s), wherein optionally the effective amount of the antibody or antigen-binding fragment thereof is administered within about 3.0 hours or within about 6.0 hours after the onset of the first stroke symptom(s).

15. The method of claim 14, wherein the effective amount of the antibody or antigen-binding fragment thereof treats stroke by increasing vascular length and/or vascular diameter of affected blood vessel(s), increasing cerebral blood flow, preventing vascular damage, recanalizing blood vessels, reducing infarct formation and/or infarct volume, inhibiting neuronal cell swelling, extending the therapeutic time window for reperfusion, reducing or preventing stroke reperfusion injury, alleviating neuroinflammation and/or reducing hypoxia-induced excitotoxicity in neurons.

16. The method of claim 15, wherein the effective amount of the antibody or antigen-binding fragment thereof extends the therapeutic time window for reperfusion from about 4.5 hours to about 5.0 hours, about 5.5 hours, about 6.0 hours or about 6.5 hours after the onset of the first stroke symptom(s), wherein optionally the effective amount of the antibody or antigen-binding fragment thereof extends the therapeutic time window for reperfusion from about 4.5 hours to about 6.0 hours after the onset of the first stroke symptom(s).

17. The method of claim 13, wherein the effective amount of the antibody or antigen-binding fragment thereof treats stroke by reducing one or more symptoms of stroke in the subject, wherein optionally the symptom of stroke is selected from the group consisting of confusion, severed numbness or weakness to one side or part of the body, severe headache, vision impairment, dizziness, walking difficulties, loss of balance or coordination, and slurred speech.

18. The method of claim 1, wherein the subject is a stroke patient who is ineligible for reperfusion therapy, wherein optionally the reperfusion therapy includes thrombolysis and/or thrombectomy.

19. The method of claim 1, further comprising administering to the subject one or more interventions selected from the group consisting of:
(a) one or more surgical interventions;
(b) one or more therapeutic agents;
(c) one or more surgical interventions selected from the group consisting of thrombectomy, carotid endarterectomy, and craniotomy; and
(d) one or more therapeutic agents selected from the group consisting of a thrombolytic agent, antiplatelets, and anticoagulants.

20. The method of claim 19, wherein the thrombolytic agent is selected from the group consisting of tissue plasminogen activator (tPA), streptokinase, urokinase, and anistreplase, wherein optionally the thrombolytic agent is tPA.


SEQUENCE LISTING (63 sequences)

SEQ ID NO: 1 - protein, 11 aa, synthetic construct
GYSITSDYAWN

SEQ ID NO: 2 - protein, 16 aa, synthetic construct
YLSYSGVTSYNPSLKG

SEQ ID NO: 3 - protein, 16 aa, synthetic construct
KGTYYRYEGSYWYFDV

SEQ ID NO: 4 - protein, 11 aa, synthetic construct
HANQNIDVWLS

SEQ ID NO: 5 - protein, 7 aa, synthetic construct
KTSNLHT

SEQ ID NO: 6 - protein, 9 aa, synthetic construct
QQGQSYPLT

SEQ ID NO: 7 - protein, 125 aa, synthetic construct
QVQLQESGPGLVKPSQTLSLTCTVSGYSITSDYAWNWIRQHPGKGLEWIGYLSYSGVTSYNPSLKGRVTISVDTSKNQFSLKLSSVTAADTAVYYCARKGTYYRYEGSYWYFDVWGQGTTVTVSS

SEQ ID NO: 8 - protein, 125 aa, synthetic construct
QVQLQESGPGLVKPSQTLSLTCTVSGYSITSDYAWNWIRQHPGKGLEWMGYLSYSGVTSYNPSLKGRVTISRDTSKNQFSLKLSSVTAADTAVYYCARKGTYYRYEGSYWYFDVWGQGTTVTVSS

SEQ ID NO: 9 - protein, 125 aa, synthetic construct
QVQLQESGPGLVKPSQTLSLTCTVSGYSITSDYAWNWIRQHPGKGLQWMGYLSYSGVTSYNPSLKGRITISRDTSKNQFSLKLSSVTAADTAVYYCARKGTYYRYEGSYWYFDVWGQGTTVTVSS

SEQ ID NO: 10 - protein, 125 aa, synthetic construct
QVQLQESGPGLVKPSQTLSLTCTVSGYSITSDYAWNWIRQHPGKGLQWMGYLSYSGVTSYNPSLKGRITISRDTSKNQFFLKLSSVTAADTAVYYCARKGTYYRYEGSYWYFDVWGQGTTVTVSS

SEQ ID NO: 11 - protein, 107 aa, synthetic construct
DIQMTQSPSSLSASVGDRVTITCHANQNIDVWLSWYQQKPGKAPKLLIYKTSNLHTGVPSRFSGSGSGTDFTFTISSLQPEDIATYYCQQGQSYPLTFGGGTKLEIK

SEQ ID NO: 12 - protein, 107 aa, synthetic construct
DIQMTQSPSSLSASVGDRVTITCHANQNIDVWLSWYQQKPGKAPKLLIYKTSNLHTGVPSRFSGSGSGTDFTLTISSLQPEDIATYYCQQGQSYPLTFGGGTKLEIK

SEQ ID NO: 13 - protein, 107 aa, synthetic construct
DIQMTQSPSSLSASVGDRVTITCHANQNIDVWLSWYQQKPGNAPKLLIYKTSNLHTGVPSRFSGSGSGTDFTFTISSLQPEDIATYYCQQGQSYPLTFGGGTKLEIK

SEQ ID NO: 14 - protein, 107 aa, synthetic construct
DIQMTQSPSSLSASVGDRVTITCHANQNIDVWLSWYQQKPGNAPKLLIYKTSNLHTGVPSRFSGSGSGTDFTLTISSLQPEDIATYYCQQGQSYPLTFGGGTKLEIK

SEQ ID NO: 15 - protein, 21 aa, synthetic construct
RDSDSNCSSEPGFWAHPPGAQ

SEQ ID NO: 16 - protein, 4 aa, synthetic construct
EPGF

SEQ ID NO: 17 - DNA, 1428 bp, synthetic construct
  source 1..1428: mol_type = other DNA
atgggctggtcatgcattattctgtttctggtcgcaactgctacaggcgtgcatagtcaagtgcagctgcaggagagcggacctggcctggtgaagccttctcagaccctgagcctcacctgtaccgtgtccggatatagcatcacaagcgactacgcctggaattggatcagacagcacccaggcaagggcctggaatggatcggctacctgtcctacagcggcgtgacatcttataaccccagcctgaaaggaagagtgaccatcagcgtggacacctctaagaaccagttcagcctgaagctgtctagcgtcacagccgctgataccgccgtgtactactgcgccagaaagggcacctactaccggtacgagggcagctactggtacttcgacgtgtggggccagggcacaaccgtgaccgtttccagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 18 - DNA, 1428 bp, synthetic construct
  source 1..1428: mol_type = other DNA
atgggctggtcatgcattattctgtttctggtcgcaactgctacaggcgtgcatagtcaagtgcagctgcaggagagcggacctggcctggtgaagccatctcagaccctgtctctcacatgtaccgtgtctggatatagcatcaccagcgactacgcctggaactggatccggcagcaccccggcaagggcctggaatggatgggctacctgtcctacagcggagttacatcttataatcctagcctgaaaggcagagtgaccatcagcagagatacctccaagaaccagttcagcctgaagctgagcagcgtgacagccgctgacaccgccgtgtactactgcgccagaaagggcacatactaccggtacgagggcagctactggtacttcgacgtgtggggccagggcaccaccgtcacagtgtccagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 19 - DNA, 1428 bp, synthetic construct
  source 1..1428: mol_type = other DNA
atgggctggtcatgcattattctgtttctggtcgcaactgctacaggcgtgcatagtcaagtgcagctgcaggagtctggacctggactggtgaagcctagccagaccctgagcctgacctgtaccgtctccggctacagcatcacctccgactacgcctggaattggatcagacagcaccccggcaagggcctccagtggatgggctacctgtcttattctggagtgacatcttacaacccaagcctgaaaggcagaatcaccatcagccgggacaccagcaagaaccagttcagcctgaagctgagcagcgtgaccgccgctgatacagccgtgtactactgcgccagaaagggcacctactaccggtacgaaggcagctattggtacttcgacgtgtggggccagggcacaacagtgaccgtgtccagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 20 - DNA, 1428 bp, synthetic construct
  source 1..1428: mol_type = other DNA
atgggctggtcatgcattattctgtttctggtcgcaactgctacaggcgtgcatagtcaagtgcagctgcaggagagcggacctggcctggtgaagcctagccagaccctgtccctgacatgtaccgtgtctggctacagcatcacaagcgactacgcctggaattggatccggcagcacccaggaaagggcctccagtggatgggctatctgagctatagcggcgtgacatcctacaaccccagcctgaaaggaagaatcaccatcagcagagatacatctaagaaccagtttttcctgaagctgtctagcgtgaccgccgctgacaccgccgtgtactactgcgccagaaagggcacctactaccggtacgaaggcagctactggtacttcgacgtgtggggccagggcacaaccgtcaccgtgtccagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 21 - DNA, 705 bp, synthetic construct
  source 1..705: mol_type = other DNA
atgggctggtcatgtattattctgtttctggtcgcaactgctacaggggtccatagtgatattcagatgacccagagccccagcagcctctccgccagcgtgggagatagagtgaccatcacctgtcacgccaaccagaatatcgacgtctggctgagctggtatcagcagaaacctggcaaggcccctaagctgctgatctacaagaccagcaacctgcacaccggcgtgccatctcggttcagcggctctggcagcggcaccgacttcacatttacaatcagctctctgcagcctgaggacatcgctacatactactgccagcaaggccagtcctaccccctgaccttcggcggaggcacaaagctggaaatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

SEQ ID NO: 22 - DNA, 705 bp, synthetic construct
  source 1..705: mol_type = other DNA
atgggctggtcatgtattattctgtttctggtcgcaactgctacaggggtccatagtgatattcagatgacccagtccccaagcagcctctccgccagcgtgggcgatagagtgaccatcacctgtcacgccaaccagaatatcgacgtgtggctgagctggtaccagcaaaagcccggcaaagcccctaagctgctgatctacaagaccagcaacctgcacaccggcgtccccagccggttcagcggatctggcagcggcaccgacttcaccctgacaatcagctctctgcagcctgaggacatcgctacatactactgccagcagggccagtcttatcctctgacatttggcggcggaacaaagctggaaatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

SEQ ID NO: 23 - DNA, 705 bp, synthetic construct
  source 1..705: mol_type = other DNA
atgggctggtcatgtattattctgtttctggtcgcaactgctacaggggtccatagtgatattcagatgacccagagccccagcagcctgtccgccagcgtcggcgatagagtgacaatcacctgtcacgccaaccagaatatcgacgtgtggctgtcttggtatcagcaaaaacctggcaacgctcctaagctcctgatctacaagaccagcaacctgcacacaggcgtgccaagccggttcagcggcagcggatctggcaccgacttcaccttcacaatcagctctctgcagcctgaggacatcgccacctactactgccagcagggccagtcctaccccctgacctttggcggaggcacaaagctggaaatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

SEQ ID NO: 24 - DNA, 705 bp, synthetic construct
  source 1..705: mol_type = other DNA
atgggctggtcatgtattattctgtttctggtcgcaactgctacaggggtccatagtgatattcagatgacccagtctccatctagcctctccgccagcgtgggcgaccgggtgaccatcacctgtcacgccaaccagaacatcgacgtgtggctgagctggtatcagcagaaacctggaaatgcccctaagctgctgatctacaagaccagcaacctgcacaccggcgtccccagcagattcagcggcagcggctctggcaccgacttcaccctgacaatcagcagcctgcagcctgaggatatcgctacatactactgccagcaaggccagtcctaccccctgacatttggcggcggaacaaagctggaaatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

SEQ ID NO: 25 - protein, 125 aa, synthetic construct
QVQLQESGPGLVKPSQSLSLTCTVTGYSITSDYAWNWIRQFPGNKLQWMGYLSYSGVTSYNPSLKGRISITRDTSKNQFFLQLSSVTPEDTATYYCARKGTYYRYEGSYWYFDVWGAGTTVTVSS

SEQ ID NO: 26 - protein, 107 aa, synthetic construct
DIQMNQSPSSLSASLGDTITITCHANQNIDVWLSWYQQKPGNVPKLLIYKTSNLHTGVPSRFSGSGSGTDFTLTISSLQPEDIATYYCQQGQSYPLTFGGGTKLEIK

SEQ ID NO: 27 - DNA, 1428 bp, synthetic construct
  source 1..1428: mol_type = other DNA
atgggctggtcatgcattattctgtttctggtcgcaactgctacaggcgtgcatagtcaggtgcagctgcaggagagcggacctggcctggtgaagcctagccagagcctgtccctcacctgtaccgtgaccggctactccatcacaagcgactacgcctggaattggatcagacagtttccaggcaacaagctgcagtggatgggctacctgtcttattctggcgtgacatcttacaaccccagcctgaaaggcagaatcagcatcacccgggacaccagcaagaaccagttcttcctgcaactgagcagcgtgacccctgaagataccgccacatactactgcgccagaaagggcacctactaccggtacgagggcagctattggtacttcgacgtgtggggcgctggaacaaccgtcacagtgtccagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 28 - DNA, 705 bp, synthetic construct
  source 1..705: mol_type = other DNA
atgggctggtcatgtattattctgtttctggtcgcaactgctacaggggtccatagtgatatccagatgaaccagagcccttctagcctctccgccagcctgggcgacaccattacaatcacctgtcacgctaatcagaacatcgacgtgtggctgagctggtatcagcagaaacctggcaacgtgcccaagctgctgatctacaagaccagcaacctgcacaccggcgtgccatctagattcagcggatctggcagcggcaccgacttcaccctgaccatcagcagcctgcagcctgaggatatcgccacatactactgccagcaaggccagtcctaccccctgacatttggcggcggaacaaagctggaaatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

SEQ ID NO: 29 - protein, 25 aa, synthetic construct
QVQLQESGPGLVKPSQSLSLTCTVT

SEQ ID NO: 30 - protein, 14 aa, synthetic construct
WIRQFPGNKLQWMG

SEQ ID NO: 31 - protein, 32 aa, synthetic construct
RISITRDTSKNQFFLQLSSVTPEDTATYYCAR

SEQ ID NO: 32 - protein, 11 aa, synthetic construct
WGAGTTVTVSS

SEQ ID NO: 33 - protein, 25 aa, synthetic construct
QVQLQESGPGLVKPSQTLSLTCTVS

SEQ ID NO: 34 - protein, 14 aa, synthetic construct
WIRQHPGKGLEWIG

SEQ ID NO: 35 - protein, 32 aa, synthetic construct
RVTISVDTSKNQFSLKLSSVTAADTAVYYCAR

SEQ ID NO: 36 - protein, 11 aa, synthetic construct
WGQGTTVTVSS

SEQ ID NO: 37 - protein, 14 aa, unidentified
WIRQHPGKGLEWMG

SEQ ID NO: 38 - protein, 32 aa, synthetic construct
RVTISRDTSKNQFSLKLSSVTAADTAVYYCAR

SEQ ID NO: 39 - protein, 14 aa, synthetic construct
WIRQHPGKGLQWMG

SEQ ID NO: 40 - protein, 32 aa, synthetic construct
RITISRDTSKNQFSLKLSSVTAADTAVYYCAR

SEQ ID NO: 41 - protein, 32 aa, synthetic construct
RITISRDTSKNQFFLKLSSVTAADTAVYYCAR

SEQ ID NO: 42 - protein, 23 aa, synthetic construct
DIQMNQSPSSLSASLGDTITITC

SEQ ID NO: 43 - protein, 15 aa, synthetic construct
WYQQKPGNVPKLLIY

SEQ ID NO: 44 - protein, 32 aa, synthetic construct
GVPSRFSGSGSGTDFTLTISSLQPEDIATYYC

SEQ ID NO: 45 - protein, 10 aa, synthetic construct
FGGGTKLEIK

SEQ ID NO: 46 - protein, 23 aa, synthetic construct
DIQMTQSPSSLSASVGDRVTITC

SEQ ID NO: 47 - protein, 15 aa, synthetic construct
WYQQKPGKAPKLLIY

SEQ ID NO: 48 - protein, 32 aa, synthetic construct
GVPSRFSGSGSGTDFTFTISSLQPEDIATYYC

SEQ ID NO: 49 - protein, 15 aa, synthetic construct
WYQQKPGNAPKLLIY

SEQ ID NO: 50 - protein, 474 aa, synthetic construct
MGWSCIILFLVATATGVHSQVQLQESGPGLVKPSQTLSLTCTVSGYSITSDYAWNWIRQHPGKGLEWIGYLSYSGVTSYNPSLKGRVTISVDTSKNQFSLKLSSVTAADTAVYYCARKGTYYRYEGSYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 474 aa, synthetic construct
MGWSCIILFLVATATGVHSQVQLQESGPGLVKPSQTLSLTCTVSGYSITSDYAWNWIRQHPGKGLEWMGYLSYSGVTSYNPSLKGRVTISRDTSKNQFSLKLSSVTAADTAVYYCARKGTYYRYEGSYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 474 aa, synthetic construct
MGWSCIILFLVATATGVHSQVQLQESGPGLVKPSQTLSLTCTVSGYSITSDYAWNWIRQHPGKGLQWMGYLSYSGVTSYNPSLKGRITISRDTSKNQFSLKLSSVTAADTAVYYCARKGTYYRYEGSYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 474 aa, synthetic construct
MGWSCIILFLVATATGVHSQVQLQESGPGLVKPSQTLSLTCTVSGYSITSDYAWNWIRQHPGKGLQWMGYLSYSGVTSYNPSLKGRITISRDTSKNQFFLKLSSVTAADTAVYYCARKGTYYRYEGSYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 474 aa, synthetic construct
MGWSCIILFLVATATGVHSQVQLQESGPGLVKPSQTLSLTCTVSGYSITSDYAWNWIRQHPGKGLEWMGYLSYSGVTSYNPSLKGRVTISRDTSKNQFSLKLSSVTAADTAVYYCARKGTYYRYEGSYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 474 aa, synthetic construct
MGWSCIILFLVATATGVHSQVQLQESGPGLVKPSQTLSLTCTVSGYSITSDYAWNWIRQHPGKGLEWMGYLSYSGVTSYNPSLKGRVTISRDTSKNQFSLKLSSVTAADTAVYYCARKGTYYRYEGSYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 471 aa, synthetic construct
MGWSCIILFLVATATGVHSQVQLQESGPGLVKPSQTLSLTCTVSGYSITSDYAWNWIRQHPGKGLEWMGYLSYSGVTSYNPSLKGRVTISRDTSKNQFSLKLSSVTAADTAVYYCARKGTYYRYEGSYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 233 aa, synthetic construct
MGWSCIILFLVATATGVHSDIQMTQSPSSLSASVGDRVTITCHANQNIDVWLSWYQQKPGKAPKLLIYKTSNLHTGVPSRFSGSGSGTDFTFTISSLQPEDIATYYCQQGQSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 233 aa, synthetic construct
MGWSCIILFLVATATGVHSDIQMTQSPSSLSASVGDRVTITCHANQNIDVWLSWYQQKPGKAPKLLIYKTSNLHTGVPSRFSGSGSGTDFTLTISSLQPEDIATYYCQQGQSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 233 aa, synthetic construct
MGWSCIILFLVATATGVHSDIQMTQSPSSLSASVGDRVTITCHANQNIDVWLSWYQQKPGNAPKLLIYKTSNLHTGVPSRFSGSGSGTDFTFTISSLQPEDIATYYCQQGQSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 233 aa, synthetic construct
MGWSCIILFLVATATGVHSDIQMTQSPSSLSASVGDRVTITCHANQNIDVWLSWYQQKPGNAPKLLIYKTSNLHTGVPSRFSGSGSGTDFTLTISSLQPEDIATYYCQQGQSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - DNA, 1425 bp, synthetic construct
  source 1..1425: mol_type = other DNA
atgggctggtcatgcattattctgtttctggtcgcaactgctacaggcgtgcatagtcaagtgcagctgcaggagagcggacctggcctggtgaagccatctcagaccctgtctctcacatgtaccgtgtctggatatagcatcaccagcgactacgcctggaactggatccggcagcaccccggcaagggcctggaatggatgggctacctgtcctacagcggagttacatcttataatcctagcctgaaaggcagagtgaccatcagcagagatacctccaagaaccagttcagcctgaagctgagcagcgtgacagccgctgacaccgccgtgtactactgcgccagaaagggcacatactaccggtacgagggcagctactggtacttcgacgtgtggggccagggcaccaccgtcacagtgtccagcgccagcacaaagggccctagcgtgtttccactggccccctcctctaagtccacctctggaggaacagccgccctgggctgtctggtgaaggattatttcccagagcccgtgaccgtgtcctggaactctggcgccctgaccagcggagtgcacacatttccagccgtgctgcagagctccggactgtactccctgtctagcgtggtgaccgtgccttcctctagcctgggcacccagacatatatctgcaacgtgaatcacaagccctccaatacaaaggtggacaagaaggtggagcctaagtcttgtgataagacccacacatgccccccttgtcctgcaccagaggcagcaggaggaccttccgtgttcctgtttccacccaagccaaaggacaccctgatgatcagccgcacccctgaggtgacatgcgtggtggtggacgtgtcccacgaggatccagaggtgaagtttaactggtacgtggatggcgtggaggtgcacaatgccaagaccaagcctcgggaggagcagtacaactctacctatagagtggtgagcgtgctgacagtgctgcaccaggactggctgaacggcaaggagtataagtgcaaggtgtctaataaggccctgcccgcccctatcgagaagaccatcagcaaggcaaagggacagcctagggagccacaggtgtacacactgcctccatctagagacgagctgaccaagaaccaggtgagcctgacatgtctggtgaagggcttctatccaagcgatatcgccgtggagtgggagtccaatggccagcccgagaacaattacaagaccacaccccctgtgctggacagcgatggctccttctttctgtattccaagctgaccgtggataagtctcggtggcagcagggcaacgtgtttagctgttccgtgatgcacgaggccctgcacaatcactacacccagaagtctctgagcctgtcccccggcaagtga

SEQ ID NO: 62 - DNA, 1425 bp, synthetic construct
  source 1..1425: mol_type = other DNA
atgggctggtcatgcattattctgtttctggtcgcaactgctacaggcgtgcatagtcaagtgcagctgcaggagagcggacctggcctggtgaagccatctcagaccctgtctctcacatgtaccgtgtctggatatagcatcaccagcgactacgcctggaactggatccggcagcaccccggcaagggcctggaatggatgggctacctgtcctacagcggagttacatcttataatcctagcctgaaaggcagagtgaccatcagcagagatacctccaagaaccagttcagcctgaagctgagcagcgtgacagccgctgacaccgccgtgtactactgcgccagaaagggcacatactaccggtacgagggcagctactggtacttcgacgtgtggggccagggcaccaccgtcacagtgtccagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacgcctctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtga

SEQ ID NO: 63 - DNA, 1416 bp, synthetic construct
  source 1..1416: mol_type = other DNA
atgggctggtcttgtattattctgtttctggtcgcaactgctacaggcgtgcattctcaagtgcagctgcaggagagcggacctggcctggtgaagccatctcagaccctgtctctcacatgtaccgtgtctggatatagcatcaccagcgactacgcctggaactggatccggcagcaccccggcaagggcctggaatggatgggctacctgtcctacagcggagttacatcttataatcctagcctgaaaggcagagtgaccatcagcagagatacctccaagaaccagttcagcctgaagctgagcagcgtgacagccgctgacaccgccgtgtactactgcgccagaaagggcacatactaccggtacgagggcagctactggtacttcgacgtgtggggccagggcaccaccgtcacagtgtccagcgccagcaccaagggcccttccgtgtttcccctggccccttgctcccggtccacatctgagagcaccgccgccctgggctgtctggtgaaggactacttcccagagcccgtgaccgtgagctggaacagcggcgccctgacaagcggcgtgcacacatttcccgccgtgctgcagagctccggcctgtactccctgtctagcgtggtgacagtgccttcctctagcctgggcaccaagacatatacctgtaacgtggaccacaagccaagcaataccaaggtggataagcgggtggagtctaagtacggccctccttgccctccatgtcctgctccagagtttctgggcggcccttccgtgttcctgtttccacccaaaccaaaggacacactgatgatctctagaacaccagaggtgacctgcgtggtggtggacgtgagccaggaggatcccgaggtgcagttcaactggtacgtggatggcgtggaggtgcacaatgccaagaccaagccaagagaggagcagtttaactctacatacagggtggtgagcgtgctgaccgtgctgcaccaggattggctcaacggcaaggagtataagtgcaaggtgtccaataagggcctgccctcctctatcgagaagacaatctctaaggctaagggccagccaagagagcctcaggtgtacaccctgcctccaagccaggaggagatgacaaagaaccaggtgtccctgacatgtctggtgaagggcttctatccctccgacatcgccgtggagtgggagtctaatggccagcctgagaacaattacaagaccacaccccctgtgctggactctgatggcagcttctttctgtattccaggctgaccgtggataagtctcggtggcaggagggcaacgtgttcagctgctctgtgatgcacgaagccctgcataatcactatactcagaaaagtctgtcactgtcactgggaaagtga